(19) United States
(12) Patent Application Publication
(10) Pub. No.: US 2004/0002465 A1
(43) Pub. Date: Jan. 1, 2004

(54) Polyene macrolide derivatives, use for vectoring molecules

(75) Inventors: Jacques Bolard, Meudon (FR); Christine Garcia, Paris (FR); Olivier Seksek, Morsang-Sur-Orge (FR); Edward Borowski, Gdansk (PL); Jolanta Grzybowska, Gdansk (PL)

Correspondence Address: YOUNG & THOMPSON, 745 SOUTH 23RD STREET 2ND FLOOR, ARLINGTON, VA, 22202

(73) Assignees: UNIVERSITE PIERRE ET MARIE CURIE, PARIS CEDEX (FR); TECHNICAL UNIVERSITY OF GDANSK, GDANSK (PL)

(21) Appl. No.: 10/373,195

(22) Filed: Feb. 26, 2003

Related U.S. Application Data
(63) Continuation of application No. 09/680,976, filed on Oct. 6, 2000, now abandoned.
Application No. 09/680,976 is a continuation of application No. PCT/FR99/00808, filed on Apr. 7, 1999.

(30) Foreign Application Priority Data
Apr. 7, 1998 (FR) 98/04317

(51) Int. Cl. A61K 48/00; A61K 31/7048

(52) U.S. Cl. 514/29; 514/44

(57) ABSTRACT

A composition having a negatively charged molecule and a cationic polyene macrolide compound having two to four positive charges that reacts with the negatively charged molecule is described. This compound can be used to vector molecules and especially nucleic acids into cells.

The present invention relates to the field of biology, in particular the transfer of compounds into cells. More particularly, it relates to novel vectors and compositions for transferring molecules of interest, in particular nucleic acids, into cells in vitro, ex vivo or in vivo. The present invention has many applications in the experimental biology, clinical or medical fields.

The possibility of effective transfer of molecules of interest into cells constitutes a major challenge in the development of biology and biotechnology. In vitro, such transfer can enable biological or biochemical experiments to be carried out (study of regulation of gene expression, mutagenesis, plasmid creation, genome studies, etc.), the production of recombinant peptides or proteins, it can produce pharmaceutical or agroalimentary interest, or it can produce recombinant viruses. Ex vivo or in vivo, such transfer allows labelling studies, bioavailability studies and tissue expression studies to be carried out; it can also be used to create transgenic animals expressing foreign genes, for example, or it can have biological applications or medical applications (vaccinations, therapies, etc.).

Thus it is of particular importance to have at one's disposal an effective system for transferring molecules of interest, applicable to populations of cells, which is non toxic to the cells or organisms used.

Different approaches have been developed in the prior art for transferring molecules of interest into cells. They concern, for example, vectors of viral origin, which are remarkably effective in transferring nucleic acids into cells. However, that type of vector also has certain potential disadvantages, primarily linked to their preparation, safety, cloning capacity, etc.

In parallel to such viral vectors, many synthetic systems have been proposed for selective delivery (i.e., transfer) of molecules of interest, in particular nucleic acids. Such synthetic systems are usually cationic so as to interact with negatively charged nucleic acids, (Legendre, 1996). However, in the majority of cases (polysine, cationic polymers, polyamines, etc.), it is considered that nucleic acid/vector complexes penetrate by endocytosis, which causes the problem (i) of subsequent leaching of nucleic acids from the endocytosis vesicles to allow them to reach the target and (ii) of degradation of nucleic acids into those cellular compartments. Further, the effectiveness of such systems in vitro is not always satisfactory.

The addition of different adjuvants to the initial formulations has been proposed to overcome that problem, so as to short-circuit endocytosis and allow direct liberation of the nucleic acid into the cytoplasm. In general, the authors envisage an entry mechanism by fusion of those complexes with endosomal or plasmic membranes (or by transient destabilisation of those membranes). Fusiogenic peptides or dioleoylphosphatidylcholine (DOPE), for example, are routinely used. Recently, effectors specifically inducing membrane permeability have been envisaged. These first concern gramicidin S (or tyrocidine) associated with DOPE vesicles (Legendre and Szoka, 1993), which formulation is inactive in the presence of serum. A peptide which disturbs membrane permeability at low pH is also found, associated with a further amphiphilic peptide (Ohmori, BBRC, 1997). This formulation is less active than Lipofectine®, a reference compound, and has non negligible toxicity.

Those latter formulations also have the disadvantage of having several components, which produces heterogeneous mixtures that are unstable in biological fluids.

While keeping the concept of acting on membrane permeability, single-component systems include a cationic amphipatic peptide (Wyman, 1997) which can cause trans-membrane leakage at low pH encountered in endosomes. Further, dioleoylmelittin (Legendre, 1997) has the advantage of being active in the presence of serum.

Despite considerable effort, the systems described in the prior art systems have disavantages linked to low in vivo activity, secondary in vivo effects, narrow ranges of activity in vitro, and/or prohibitive costs.

The present invention concerns a novel system for selective delivery of molecules of interest into cells. The system of the invention is adapted to selective delivery (transfer) of negatively charged molecules, more particularly nucleic acids, into cells.

More particularly, the invention resides in the development and/or use of compounds comprising a cationic portion that can interact with negatively charged molecules of interest, and a particular active portion which allows transfer into cells. More particularly, the compounds used in the present invention are cationic polyene macrolide compounds comprising polyene macrolide antibiotics and derivatives (in particular cationic derivatives) thereof.

The compositions of the invention have the advantage of being simple to prepare, of low toxicity, with conservation of activity in the presence of serum, with good effectiveness, and of compatibility with pharmacological use.

The present invention also describes novel polyene macrolide antibiotic molecules (such as amphotericin B) for use in selective delivery of molecules of interest or as antifungal agents.

The present invention thus provides an alternative to the systems described in the prior art for selective delivery of molecules of interest.

Thus in a first aspect, the invention provides a composition comprising:

a negatively charged molecule of interest; and

a cationic polyene macrolide compound which is capable of interaction with said molecule.

As indicated above, the invention concerns compounds or compositions allowing transfer of “molecules of interest” into cells. Essentially, they are negatively charged molecules, taking into account the cationic nature of the selective delivery compounds used. Examples of negatively charged molecules which can be cited are proteins, peptides or polypeptides, PNAs or nucleic acids.

Advantageously, they are nucleic acids. The nucleic acids can be deoxyribonucleic acids (DNA) or ribonucleic acids (RNA). Regarding DNA, complementary DNA, genomic DNA, synthetic DNA or semi-synthetic DNA can be cited. The RNA can be messenger RNA, transfer RNA, ribosomal RNA or synthetic RNA. The nucleic acids can be of human, animal, vegetable, viral, bacterial or artificial origin, for example. They may be oligonucleotides (between 3 and 80 mers long) or coding phrases, genes, genomic regions or entire chromosomes. They may be single or double-stranded nucleic acids. In particular, they may be antisense (or anti-gene) nucleic acids, i.e., capable of interfering, by hybridisation, with the activity of a gene or RNA or a particular genomic region. They may also be nucleic acids comprising a region coding for a peptide product of interest (polypeptide or protein with biological activity of immunological, pharmaceutical or alimentary interest). Further, these nucleic acids can be in the form of a linear or circular plasmid.

The nucleic acids can be obtained using any technique which is known to the skilled person, such as artificial synthesis, screening of libraries, isolation from plasmids, etc., or any combination of those conventional techniques or any other molecular biological technique.

The negatively charged molecule can also be a protein, a polypeptide or a peptide, in particular an antigenic peptide.

As indicated above, the system of the invention advantageously resides in the use of polyene macrolide antibiotics and derivatives (in particular cationic derivatives) thereof.

The polyene macrolide antibiotic family essentially comprises two groups: aromatic polyene macrolide compounds and non aromatic polyene macrolide compounds. The first are often isolated from micro-organisms in a form carrying a certain net positive charge at neutral pH. The following heptaene macrolide compounds can be cited as examples: hamycin, trichomycin, candicin, vacidin A (syn. partricin B), gedamycin (syn. partricin A) or perimycin, in particular perimycin A. Compounds of the second group (non aromatic) are zwitterionic compounds, and thus neutral overall, with the exception of lienomycin. Amphotericin B belongs to the second group, in particular of amphotericin B, amphotericin B is currently clinically used as an antifungal agent in the form of Fungizone® (amphotericin B associated with deoxycholate). Amphotericin B is also known to interact with membranes containing sterols to form, in a sub-lethal concentration, trans-membrane pores both in fungal cells and, in a higher concentration, in mammalian cells (Hartsel and Bolard, 1996).

However, the use of these polyene macrolide compounds or cationic derivatives thereof, in particular cationic heptaenic macrolide compounds, has never been reported for selective delivery of molecules.

In particular, many polyene macrolide antibiotics are uncharged overall and thus could not produce a nucleic acid/vector complex. This is the case with amphotericin B, for example, which does not per se appear to be capable of interacting with nucleic acids. The present invention now demonstrates that it is possible to use natural, synthetic or semi-synthetic polyene macrolide compounds carrying a positively charged group to transfer negative molecules, in particular nucleic acids. In particular, the present invention demonstrates that it is possible to use derivatives of polyene macrolide compounds, such as amphotericin B, to form complexes with nucleic acids, and that these complexes can be effectively delivered into cells, i.e., that (i) an electrostatic bond is established between this macrolide compound and the negative charges of the nucleic acid, and (ii) that the polyene portion of the complex retains its membrane activity.

The present invention also demonstrates that such complexes can protect nucleic acids from degradation by serum, and can thus increase the effectiveness of transfer under serum conditions, in particular in vivo or ex vivo.

In a particular embodiment, the invention concerns a composition as defined above in which the cationic polyene macrolide compound is an aromatic heptaenic macrolide antibiotic. Advantageously, the compound is perimycin.

In a further particular embodiment of the invention, the invention concerns a composition as defined above in which the cationic polyene macrolide compound is a polyene macrolide antibiotic comprising one or more cationic functional groups.

The term “derivative” as used in the invention means any form that is chemically modified by introducing at least one cationic functional group.

Further, the expression “cationic functional group” includes any group which can become cationic by protonation by the medium used (in particular the pH of the medium) and any group carrying one or more permanent positive charges. As indicated above, the compound or compounds used in the present invention are capable of interacting with the molecule of interest due to the presence of cationic functional groups. These groups are generally covalently bonded to the polyene macrolide compound. More particularly, the cationic function of the compounds used in the invention can be composed of any positively charged group which can interact with the molecule or molecules of interest. The expression “capable of interacting” means that the cationic portion can associate with the anionic molecule of interest, in particular a nucleic acid. The interaction between the compounds and the nucleic acid is essentially a non-covalent ionic interaction of the electrostatic bond type, which is established between the positive charges of the cationic portion of the compound and the negative charges of the molecule of interest (nucleic acid). Van der Waals or hydrophobic type interactions may supplement this ionic interaction. The non-covalent character of the interaction is advantageous in that it enables the complex to dissociated in the cell and thus liberate the molecules of interest in the cells. Further, this type of bond simplifies operations, as bringing the compounds and molecules of interest into contact is sufficient to form the complexes.

In contrast to systems in which the nucleic acid is bonded directly to the active portion of the membrane effector which must therefore inhibit its specificity, the present invention describes a formulation where the nucleic acid is bonded to a vector in an electrostatic manner, but in a manner that leaves the active portion free, which keeps its effectiveness intact. Further, at the concentrations used, the permeabilising activity obtained with the compositions of the invention is doubtless transient and cannot be toxic to the cell.

More preferably, in the compositions of the invention, the compound is a heptaene macrolide antibiotic. More particularly, it is a cationic derivative of amphotericin B, candidin, mycoheptin or vacidin. It may also be a derivative of nystatin. Preferably again, the compound is a non aromatic heptaene macrolide antibiotic derivative, preferably a derivative of amphotericin B.

Preferably, the compound used carries at least two positive charges. Examples of positively charged functional groups which can be bonded to the polyene macrolide compounds are esters, (poly)amides, hydrazide, polyamines, N-alkyl, N-aminoacyl, polylysines, guanidines, or combinations thereof, or more generally any hydrocarbon group containing one or more positive charges, which may in particular be provided by nitrogen atoms. Specific examples of a cationic portion which are suitable for the compounds of the invention are alkylester groups (for example methyl-, ethyl- or propyl-ester), alkylammonium groups (in particular trimethylammonium), lysil, ornithyl, guanidino, amidic or spermidic groups.

The cationic functional group or groups can be bonded to the macrolide compound at different positions. In a first embodiment, the cationic portion is introduced to an amine function of the polyene macrolide, in particular to the amine function of the mycosamine group of the molecule. In a further embodiment, the cationic portion is introduced to the carboxyl function of the aglycone group of the molecule. It is also possible to introduce the cationic portion by modifying these two positions of the molecule (see Schaffner et al., (1987), hereby incorporated by reference, and the examples below). It should also be understood that the cationic portion can be inserted at other positions in the molecule.

In a particular embodiment of the invention, the invention concerns a composition as defined above in which the polyene macrolide antibiotic derivative comprises one or more cationic functional groups covalently bonded to the carboxyl function of the aglycone group and/or to the amine function or functions of the polyene macrolide antibiotic.

It is also understood that, in addition to the cationic functional group(s), the polyene macrolide antibiotic derivative compound of the invention can compriseother structural modification(s), provided that the compound obtained retains its activity, i.e., (i) the capacity to interact with nucleic acids and (ii) the capacity for molecule transfer. This latter property allows the compounds of the invention to deliver molecules of interest to the cells, possibly by membrane permeabilisation without utilising the endocytic route. This property is ensured in the compounds of the invention by the original use of polyene macrolides such as amphotericin B or its derivatives. Using this original active portion could advantageously induce transient permeabilisation of the cells to allow the molecules of interest to pass. The use of the compounds of the invention is thus particularly advantageous since the cells are not significantly disturbed by transfer of the molecule. This “active portion” can in particular be constituted by amphotericin B (the formula for which is shown in FIG. 1), or any variation thereof or heptaene macrolide antibiotic with modified solubility, toxicity, bioavailability and/or permeabilisation properties. Such variations have been described, for example, in Schaffner et al (1987), Malewicz et al. (1980); Binet et al (1988), Chéron et al., (1989) or Brajtburg et al. (1990), hereby incorporated by reference.

In a particularly preferred embodiment, the present invention concerns a composition comprising a negatively charged molecule of interest such as a nucleic acid, and a cationic derivative of amphotericin B.

In a first particular variation, the cationic derivative is a primary, secondary or tertiary amide of amphotericin B (see FIG. 2). More particular examples which can be cited in this regard are aminoalkyl amides such as dimethylaminopropyl amide, polyamine amides, in particular spermine, polyaminoacyl ester amides, in particular dilysil methylester, or amides of heterocyclic amies such as N-methylpiperazine, 2-pyridyl ethylamine or 2-morpholine ethylamine.

In a particular variation, the cationic derivative is an ester of amphotericin B, preferably a choline ester or a dimethylaminopropyl ester (see FIG. 3).

In a particular supplementary variation, the cationic derivative is a hydrazide of amphotericin B preferably the N-methyl piperazine hydrazide of amphotericin B (see FIG. 4).

In a still further particular variation, the cationic derivative is a N-alkyl derivative of amphotericin B, preferably a N′,N′,N′-trimethyl or N′, N′-dimethylaminopropyl succinimidyl derivative of amphotericin B methyl ester (see FIG. 5).

In a further particular variation, the cationic derivative is a N-aminoacyl derivative of amphotericin B, preferably N-ornithyl-, N-diaminopropionyl-, N-lysil-, N-hexamethyllysil-, N-tetramethyllysil-, N-piperdine-propionyl-, N-piperidine-acetyl-, N′,N′-methyl-1-piperazine-propionyl- or N,N′-methyl-1-piperazine-acetyl-amphotericin B methyl ester (see FIG. 6).

In a particular embodiment of the invention, the cationic derivative of amphotericin B comprises a cationic functional group that is simultaneously at the carboxyl and amino positions of amphotericin B (see FIG. 7). Examples of such derivatives are N-ornithyl amphotericin B dimethylamino propylamide and N-piperidino acetyl amphotericin B-(4-methyl)-piperazide.

Further, in the compositions of the invention, the above compounds can be in the form of salts such as the chloride, aspartate, glutamate or ascorbate.

In a further preferred implementation, the present invention concerns a composition comprising a negatively charged molecule of interest, such as a nucleic acid, and a cationic derivative of a heptaene macrolide antibiotic selected from nystatin, candidin and vacidin (see FIG. 8). Examples of such compounds are the amide of dimethylaminopropyl nystatin, the amide of dimethylaminopropyl candidin, the amide of dimethylaminopropyl vacidin and N′-dimethylaminoacetylvacidin-dimethylaminopropylamide.

Particular examples of preferred compounds of the invention are:

Amphotericin B dimethylaminopropylamide (AMA). AMA corresponds to compound (I) shown in FIG. 2. This compound is constituted by amphotericin B onto which a diamino cationic group (HN-(CH₂)₃-NH(CH₃)₂) has been bonded at the 16 position (carboxylic acid function of the aglycone group). This compound comprises two cationic charges.

Amphotericin B N1-sperminylamide (AMSA). AMSA corresponds to compound (II) shown in FIG. 2.

Amphotericin B (N²-lysyllysine methylester) amide (AMDLA). AMDLA corresponds to compound (III) shown in FIG. 2.

Amphotericin B B-4-methylpiperizyne amide (AMPA). AMPA corresponds to compound (IV) shown in FIG. 2.

Amphotericin B 2-(2-pyridyl)ethylamide (AMPEA). AMPEA corresponds to compound (V) shown in FIG. 2.

Amphotericin B-2-(morpholyl)ethylamide (AMMEA). AMMEA corresponds to compound (VI) shown in FIG. 2.

Amphotericin B choline ester (AMCE). AMCE corresponds to compound (VII) shown in FIG. 3.

Amphotericin B 3-dimethylaminopropyl ester (AMPE). AMPE corresponds to compound (VIII) shown in FIG. 3.

Amphotericin B methyl ester (AME). AME corresponds to compound (IX) shown in FIG. 3. This compound is constituted by amphotericin B in which the carboxylic acid function of the aglycone group has been esterified. This compound comprises one cationic charge.

The 2-dimethylaminoethyl ester of amphotericin B (AMEE). AMEE corresponds to compound (XXV) shown in FIG. 3.

Amphotericin B-4-methylpiperazine hydrazide (HAMA). HAMA corresponds to compound (X) shown in FIG. 4.

N,N,N-trimethylammonium AME (DMS-AME). DMA-AME corresponds to compound (XI) shown in FIG. 5. This compound is constituted by amphotericin B onto which two cationic groups have been grafted: a methyl ester group in the 16 position (carboxylic acid function of the aglycone group) and a trimethylammonium group in the 19 position (on the amine function of the mycosamine group). This compound comprises two cationic charges.

N-(N′-3-dimethylaminopropyl-succinimido) amphotericin B methyl ester (SAME). SAME corresponds to compound (XII) shown in FIG. 5.

N-ornithyl AME (OAME). OAME corresponds to compound (XIII) shown in FIG. 6.

N-diaminopropionyl AME. DAME corresponds to compound (XIV) shown in FIG. 6.

N-lysil-AME (LAME). LAME corresponds to compound (XV) shown in FIG. 6. This compound is constituted by amphotericin B onto which two cationic groups have been grafted: a methylester group in the 16 position (carboxylic acid function of the aglycone group) and a diamine group in the 19 position (on the amine function of the mycosamine group). This compound comprises two cationic charges.

N-(Nα, Nα, Nα,Nε, Nε, Nε,-hexamethyl) AME (MLAME). MLAME corresponds to compound (XVI) shown in FIG. 6.

N-(4-methyl-1-piperazinepropionyl)AME (PNAME). PNAME corresponds to compound (XVII) shown in FIG. 6.

N-(1-piperdinepropionyl) AME (PAME). PAME corresponds to compound (XVIII) shown in FIG. 6.

N-ornithyl-AMA (OAMA). OAMA corresponds to compound (XIX) shown in FIG. 7.

N-(4-methyl-1-piperazinepropionyl) AMPA (PAMPA). PAMPA corresponds to compound (XX) shown in FIG. 7.

Nystatin A1 3-dimethylaminopropyl amide (NYA). NYA corresponds to compound (XXI) shown in FIG. 8.

Candidin 3-dimethylaminopropyl amide (CAA). CAA corresponds to compound (XXII) shown in FIG. 8.

Vacidin A 3-dimethylaminopropyl amide (VAA). VAA corresponds to compound (XXIII) shown in FIG. 8.

N-(N′,N′-dimethylglycyl)-vacidin A 3-dimethylaminopropyl amide (VAGA). VAGA corresponds to compound (XXIV) shown in FIG. 8.

The examples will show that these positively charged compounds and in particular those with at least 2 positive charges such as AMA can produce the following results:

by interacting with the compound, the oligonucleotides are protected from degradation induced by serum;

cellular internalisation of oligonucleotides labelled with fluorescein into cells (MCF7 and 3T3) is greatly enhanced by the compound. In particular, compared with the action of a reference compound (Lipofectine®), AmA induces homogeneous rather than spotted intracellular distribution and the majority of the cell population is targeted;

expression of the MDR1 gene in fibroblasts transfected with MDR1 is greatly reduced by anti-MDR1 antisense phosphorothioate oligonucleotides delivered by the compound;

the compound can also deliver genes: using it, we have been able to transfect the GFP (green fluorescent protein) gene.

These examples show that the compounds of the invention such as AmA, have interesting vector characteristics for the transfer of nucleic acids into cells (genes, antisense or anti-gene oligodeoxyribonucleotides).

In the compositions of the invention, the respective quantities of compound and molecule(s) of interest (for example nucleic acid) are preferably selected such that the ratio (R) of the positive charges of the compound to the negative charges of the molecule is in the range 0.1 to 20. More preferably, this ratio is in the range 0.5 to 15. It is understood that this ratio can be adjusted by the skilled person as a function of the molecule of interest (in particular nucleic acid) and the compound used, the envisaged application and the target cell type.

The compositions of the invention are generally prepared by incubation (for example by contact in solution) of the compound/compounds with the molecule/molecules of interest (nucleic acids) for a period of time sufficient to allow interaction. The incubation period is also a function of the compounds used and the incubation conditions (medium, agitation, etc.). Advantageously, incubation is carried out for a period in the range 15 minutes to 2 hours, for example. The method also comprises a step for formation of a complex between the compound/s and the molecule/s (nucleic acids) which can be monitored in different manners, and in particular by following the absorption spectrum of the solution, as will be illustrated in the examples.

Incubation can be carried out in different media, preferably in the absence of serum to prevent degradation of the nucleic acids before complexing. They may be saline solutions, buffers (PBS), etc., in which the compounds/nucleic acids are soluble. Examples of suitable media are DMEM, RPMI or any medium that is compatible with in vitro, ex vivo or in vivo use. Clearly, the choice of medium can be left to the skilled person.

The compounds used in the invention can be synthesised using different possible routes that are known to the skilled person. Thus it is possible to synthesise these compounds from amphotericin B by coupling the cationic portion or portions using conventional chemical methods. In this regard, the methods described by the following can be used: Falkowski et al. (J. Antibiot. 33 (1980) 103; J. Antibiot 35 (1982) 220; J. Antibiot. 28 (1975) 244 and J. Antibiot. 31 (1979) 080), Schaffner et al., (Antibiot. Chemother. 11 (1961) 724) , Mechlinski et al., (J. Antibiot. 25 (1972) 256) or Pandey et al., (J. Antibiot. 30 (1977) 158), hereby incorporated by reference.

It is also possible to produce these compounds starting from other polyene macrolide antibiotics.

Clearly, the skilled person can adapt the preparation method using common general knowledge.

Further, the present invention relates to novel polyene macrolide antibiotic derivative compounds, in particular of amphotericin B, endowed with the capacity of delivering nucleic acids into cells and with antifungal properties. These compounds comprise a portion derived from amphotericin B or other heptaene macrolide antibiotics to which one or more positively charged groups or combinations of groups are covalently bonded, which groups have not previously been used to make chemical modifications to polyene antibiotics. They have been selected from the following groups: choline, polyamine, hydrazide, N-acyl, etc. Such compounds are represented, for example, by the products AMPEA, AMSA, AMDLA, AMPA, AMCE, AMPE, AMEE, SAME, PNAME, PAME, MLAME, OAMA, PAMPA and VAGA as defined above.

The structures of all of the derivatives are represented in the figures in the ionised form, to indicate the position and number of positive charges acquired at a physiological pH or by interaction with acids, including nucleic acids (protonation). Compounds with quaternary ammonium groups carry permanent positive charges.

The novel compounds described above also possess a high antifungal capability. As illustrated in the examples, these compounds are capable of strongly inhibiting the growth (and causing the death) of fungal cells. These compounds can thus be used as antifungal agents, in particular to induce the destruction of fungal cells in vitro, ex vivo or in vivo. In this regard, the invention thus also concerns any pharmacological (in particular pharmaceutical) use of the amphotericin B derivatives described above. More particularly, it concerns the use of these compounds as antifungal agents and any antifungal composition comprising said compound. The antifungal activity can be used for any fungal cell type preferably expressing an ergosterol group or a corresponding precursor, as will be illustrated below. The conditions for obtaining this activity (doses, time, etc.) in vitro and in vivo can readily be transposed from those described for amphotericin B, for example by using IC50s.

The compounds/compositions of the invention can be used to transfer molecules of interest into different types of cells, tissues or organs, in vitro, ex vivo or in vivo. In particular, these compounds/compositions can be used for transfer into any cell type which is sensitive to the polyene macrolide antibiotics used, in particular amphotericin B, i.e., on which the antibiotic or antibiotics exert a membrane permeabilisation activity.

Preferably, they are cells containing ergosterol groups or precursors thereof in their membrane.

Examples which can be cited are parasitic protozoa (for example leishmania) or fungal cells, the preferred target for polyene antibiotics such as amphotericin B. fungal cells which can be cited include candida, cryptococcus or aspergillus cells.

It is also possible to cite yeast cells such as Saccharomyces, Kluyveromyces, etc.

Further, the compounds of the invention are also capable of delivering molecules into somatic cells of the fibroblast, hepatic, muscle, nerve, haematopoietic, etc. type. As will be seen in the examples, these compounds are effective on fibroblasts (3T3) and on human cancer cells (MCF-7), demonstrating their large range of activity.

Clearly, when transferring molecules into cells, the dose of the compound(s) used is preferably non toxic for the cells.

In a further aspect, the invention also concerns cells modified by a composition as described above. In the context of the invention, the term “modified cell” means any cell comprising a molecule of interest delivered by a composition or a compound as defined above. As indicated above, the cells can be fungal cells, parasitic protozoa (for example leishmania) or mammalian cells, in particular human cells. The modified cells of the invention can be obtained by a method comprising incubating the cells in the presence of a composition of the invention in vitro, ex vivo or in vivo, incubation being carried out using any appropriate apparatus (plate, dish, fermenter, etc.). In vivo, incubation can be carried out by administering a composition of the invention to a subject (preferably a mammal) under conditions known to the skilled person. In particular, administration may be topical, oral, parenteral, nasal, intravenous, intramuscular, subcutaneous, intraoccular, transdermal, etc. For this type of application, the compositions of the invention advantageously comprise a physiologically acceptable vehicle, such as saline solutions (monosodium phosphate, disodium phosphate, sodium chloride, potassium chloride, calcium chloride or magnesium chloride, etc., or mixtures of such salts), sterile, isotonic, or dry compositions, in particular freeze-dried compositions which, by adding sterilised water or physiological serum depending on the case, can constitute adminstratable aqueous solutions.

The compositions of the invention have many applications and comprise, for example, the production of recombinant proteins, the study of the regulation of gene expression, labelling or bioavailability studies, the creation of non-human trangenic anaimals, or different medical applications. In this regard, the examples demonstrate the effectiveness of the compositions of the invention in the context of antisense strategies directed against resistance to antitumorals or in the context of transfer of marker genes. These results can extend to:

multiple resistance to antifungal agents: since fungal cells are the preferred target of polyene antibiotics, selective delivery of oligonucleotides or genes directed against the resistance provide an application of choice. Resistance to novel antifungal agents such as azole derivatives is beginning to appear and presents a serious threat in the future if preventative measures are not taken. In this respect, in a particular aspect, the invention concerns the use of a composition as defined above for preparing a drug intended for transfer, into a fungal cell, of a nucleic acid reducing the resistance of said cell to antibiotics.

any antisense or anti-gene approach, in particular intracellular;

the production of ex vivo or in vivo proteins, in particular proteins selected from hormones, enzymes, growth factors, trophic factors, coagulation factors, lipoproteins, lymphokines, etc.;

transfection for gene therapy.

Some advantages of the compounds/compositions of the invention are:

simple, cheap preparation;

low toxicity;

their activity in the presence of serum and antibacterial agents;

wide clinical use of Fungizone®, which will act as a reference for clinical studies on the derivatives;

their selectivity: better than the majority of membrane effectors, regarding action on membrane permeability: the formation of transient and reversible transmembrane pores and not destabilising lytic action.

The present invention will now be described in more detail with reference to the following examples, which should be considered to be illustrative and non-limiting.

### DESCRIPTION OF FIGURES

FIG. 1: Structure of amphotericin B.

FIG. 2: Structure of amide derivatives of amphotericin B.

FIG. 3: Structure of ester derivatives of amphotericin B.

FIG. 4: Structure of hydrazide derivatives of amphotericin B.

FIG. 5: Structure of N-alkyl derivatives of amphotericin B.

FIG. 6: Structure of N-aminoacyl derivatives of amphotericin B.

FIG. 7: Structure of multiple derivatives of amphotericin B.

FIG. 8: Structure of other cationic polyene macrolides.

FIG. 9: Absorption spectrum of AmA (5×10⁻⁶M) before and after adding oligonucleotide (ODN, 10⁻⁶M).

FIG. 10: Absorption spectrum of AmE (10⁻⁵M) before and after adding oligonucleotide (ODN, 1.85×10⁻⁶M).

FIG. 11: Circular dichroism of AMA (2×10⁻⁵ M) in the presence (b) and absence (a) of pGFP.

FIG. 12: Effect of AmA alone or in the presence of oligonucleotide (AS, 20 mers, 0.5 μM) on the viability of 3T3 cells treated for 4 h at 37° C.

FIG. 13: Internalisation into 3T3 cells of an oligonucleotide labelled with fluorescein (ODN, 20 mers) whether not delivered (A) or delivered by Lipofectine® (B) or by AmA (C) or by AmA (D).

FIG. 14: Internalisation into MCF-7 cells of an oligonucleotide labelled with fluoresein (ODN, 20 mers) whether not vectorised (A) or vectorised by Lipofectine® (B) or by AME (C and D).

FIG. 15: Gel autoradiograph (20% polyacrylamide-7M urea) onto which ODN is deposited in the absence or presence (+/−=10) of AmA after different incubation periods (0.4, 8 and 16 h respectively) in a Hépès buffer containing 10% (v/v) of foetal calf serum.

FIG. 16: Expression of P-glycoprotein (P-gp) in 3T3 R cells (columns 1 and 3-8) or 3T3 S cells (column 2) after forty-eight hours of treatment.

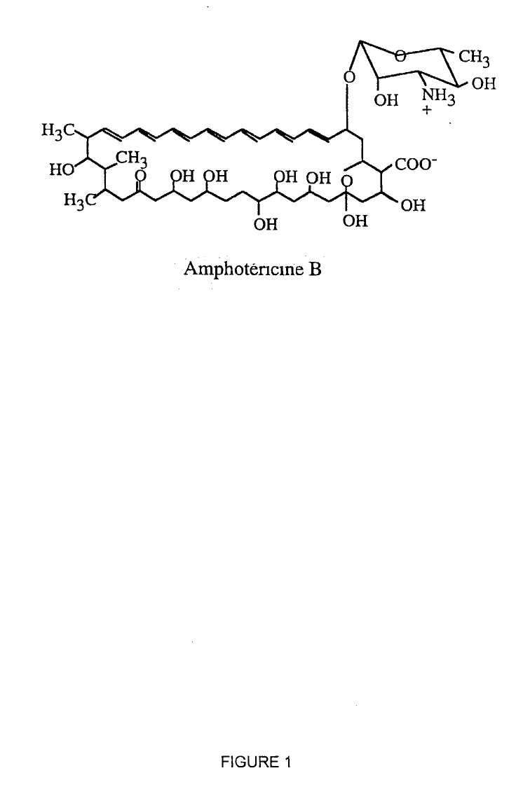

The different treatments, prepared in DMEM medium without foetal calf serum, were as follows:

Controls:

1- untreated 3T3 R;

2- untreated 3T3 S; study of antisense effect:

3- 3T3 R/AS5995 (1 μM) delivered by Lipofectine® (20 μg/ml);

4- 3T3 R/CTL (1 μM) delivered by Lipofectine® (20 μg/ml);

6- 3T3 R/AS5995 (1 μM) delivered by AMA (5×10⁻⁶ M);

7- 3T3 R/CTL (1 μM) delivered by AMA (5×10⁻⁶ M)

9- non delivered 3T3 R/AS5005 (1 μM).

Study of possible vector effect:

5- 3T3 R/Lipofectine® (20 μg/ml)

8- 3T3 R/AMA (10⁻⁵ M)

### EXAMPLE 1

Study of Absorption Spectrum of Cationic Derivatives of Amphotericin B in the Presence of Oligonucleotides

3 ml of RPMI medium free of phenol red (Gibco BRL, Life Technologies, S. A., Cergy Pontoise, France) was added to a mixture of 22 μl of AmA (stock solution in DMSO, 7×10⁻⁴ M) and 16 μl of a 20-mer oligonucleotide (Genosys Biotechnologies, The Woodland, England) (stock solution, 1.9×10⁻⁴ M in water). The solution was incubated at ambient temperature for 30 minutes. The sequence for the oligonucleotide (CCATCCCGACCTCGCGCTCC, SEQ ID NO:1) came from Alahari et al (Alahari, Dean et al., 1996) and corresponded to an antisense oligonucleotide directed against the RNA of the MDR1 gene and thus was intended to inhibit expression of the MDR1 gene.

The absorption spectra of these solutions were recorded using a Hewlett-Packard 8452A UV-visible spectrophotometer.

The spectra were also measured for a mixture of 15 μl of AmE (stock solution in water, 10⁻² M), 3 ml of PBS buffer, pH 7.4, and 28 μl of a 27-mer oligonucleotide (Genosys Biotechnologies, The Woodland, England) (stock solution, 2×10⁻⁴ M). The sequence for this oligonucleotide has been determined by Quattrone et al. (Quattrone et al., 1994) and corresponded to an anti-gene oligonucleotide directed against the MDR1 gene, i.e., an oligonucleotide capable of inhibiting expression of the MDR1 gene (5′-TGT GTT TTT GTT TTG TTG GTT TTG TTT-3′; SEQ ID NO:2).

Polyene antibiotics have specific absorption bands between 300 and 450 nm. Under the conditions of this experiment (5×10⁻⁶ M of antibiotic), free AmE or free AmA exhibit a band at 330 nm and a shoulder at 420 nm, characteristic of the self-associated formes of the antibiotic, and four bands at 345, 365, 385 and 409 nm.

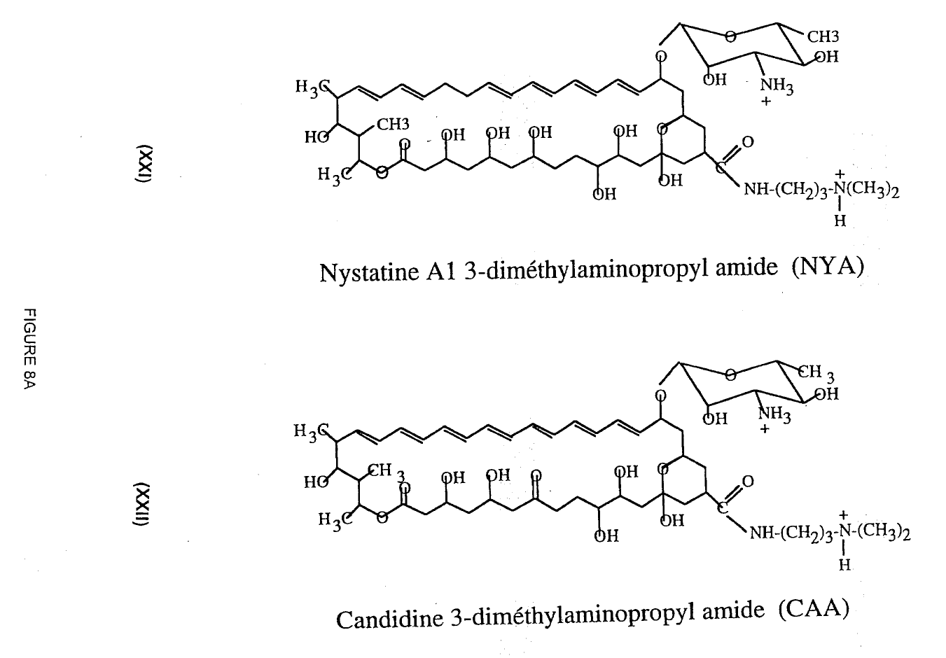

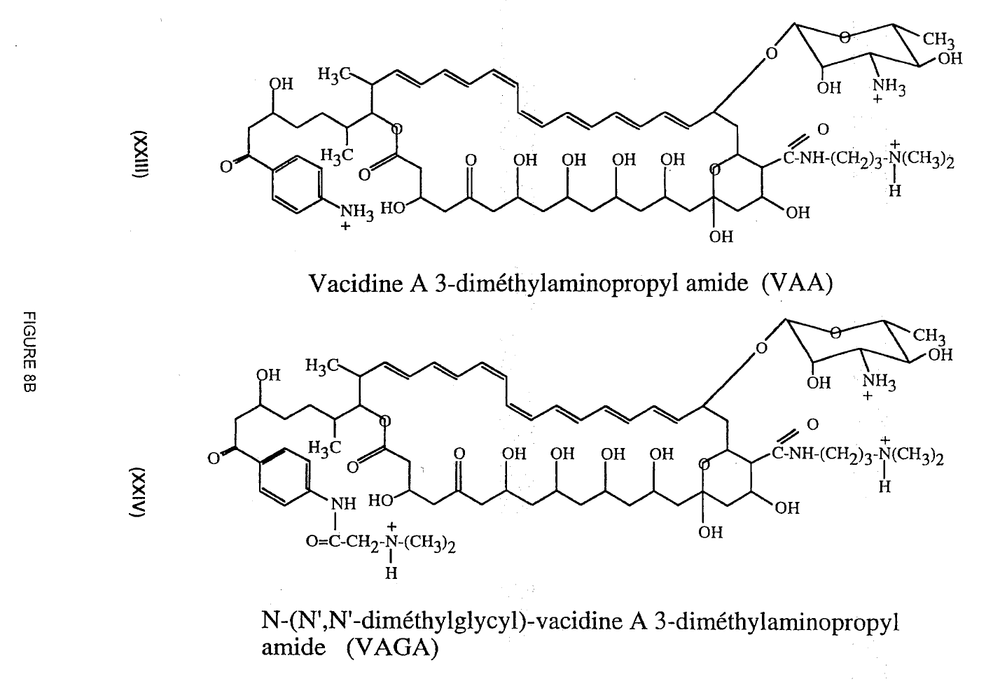

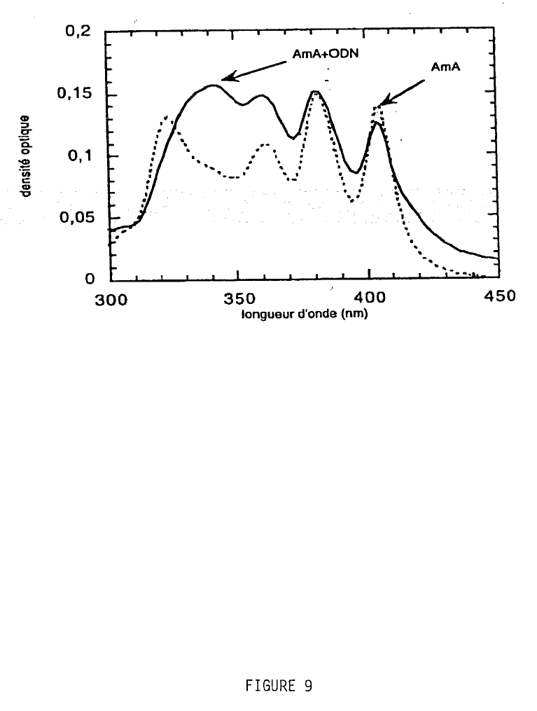

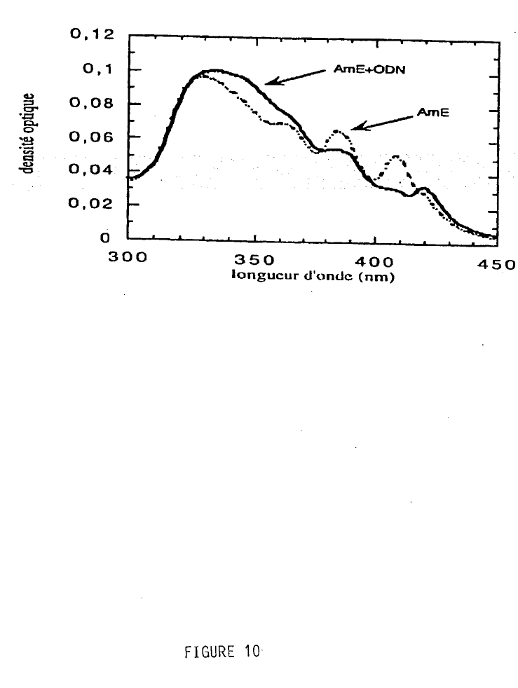

With AmA, in the presence of oligonucleotide (10⁻⁶ M), background noise appeared, the intensity of the monomer bands at 385 and 409 nm reduced, and the band at 330 nm was intensified and displaced towards the red (see FIG. 9). With AME, in the presence of oligonucleotide, the intensity of the bands at 345 and 420 nm increased while that of the bands at 409 and 385 nm decreased (see FIG. 10). In the two cases, these changes indicate that an interaction between the antibiotic and the oligonucleotide is produced and the self-association state of the antibiotic is modified. The spectrum of the oligonucleotide around 255 nm did not change, if the increase in absorption resulting from the increase in the background noise was taken into account.

### EXAMPLE 2

Circular Dichroism Spectrum of AmA in the Presence of pGFP emd-c [R] Plasmid

1.5 ml of RPMI medium free of phenol red was added to a mixture of 30 μl of a stock solution of 10⁻³ M AmA in DMSO and 6 μl of pGFP emd-c [R] plasmid (Packard, Instrument Company, Meriden, USA) (1 μg/ml). The solution was incubated at ambient temperature for 30 minutes. Circular dichroism spectra of these solutions were recorded using a Jobin-Yvon Mark V dichrograph.

Polyene antibiotics have a specific doublet at about 320 nm. In the presence of the pGFP emd-c [R] plasmid, this doublet was displaced towards the red and its intensity reduced (see FIG. 11). These characteristics indicate that an interaction between AMA and the plasmid leads to a modification in the self-association state of the antibiotic.

### EXAMPLE 3

Study of the Toxicity of AmA in the Presence of Antisense Oligonucleotides on 3T3 Cells

A 20-mer oligonucleotide was obtained from Genosys Biotechnologies (The Woodlands, England). Its sequence has been determined by Alahari et al., and corresponds to an antisense oligonucleotide that inhibits expression of the MDR1 gene (see Example 1). 3T3 fibroblasts were obtained from ATCC (American Type Culture Collection).

The toxicity was measured using solutions containing concentrations of AmA ranging from 10⁻⁶ M to 5×10⁻⁵ M in the presence of absence of oligonucleotides in a final concentration of 10⁻⁴ M. in all of the experiments, the mixture was incubated for 30 minutes at ambient temperature and prepared in a serum-free DMEM medium. The cells were then seeded in a DMEM medium and placed in 96-well microplates in an amount of 4×10⁴ cells/ml. After 24 hours, the cells were rinsed with serum-free DMEM, and 200 μl of freshly prepared AmA/oligonucleotide solution was added to the wells.

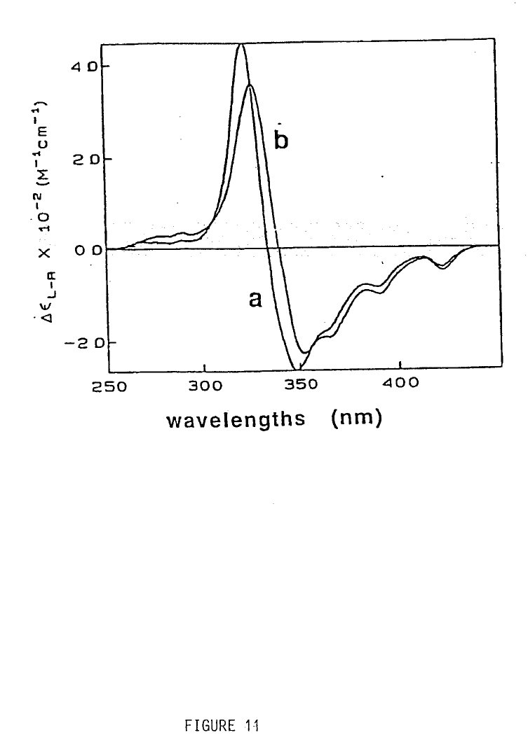

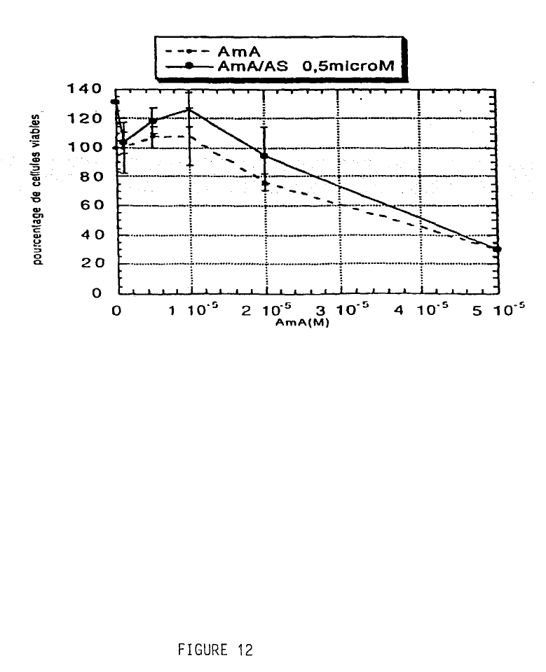

After incubating for 4 hours at 37° C., cellular viability was measured by a colorimetric test using MTT, using the methodology described in the literature (Mosmann, 1983). The results are shown in FIG. 12 and demonstrate that below 2×10⁻⁵ M, no toxicity was observed. Further, these results also show that below 10⁻⁵ M, cell growth appears to have been stimulated.

### EXAMPLE 4

Internalisation of Antisense Oligonucleotides Labelled With FITC Into 3T3 Cells

A 20-mer oligonucleotide labelled with FITC was obtained from Genosys Biotechnologies (The Woodlands, England). Its sequence has been determined by Alahari et al., and corresponds to an antisense oligonucleotide that inhibits expression of the MDR1 gene. Lipofectine was obtained from Gibco (Life Technologies, Cergy-Pontoise, France). Lipofectine® was used as the reference molecule to transfer oligonucleotides in a concentration of 20 μg/ml under the conditions recommended by the manufacturer. 3T3 fibroblasts were obtained from ATCC.

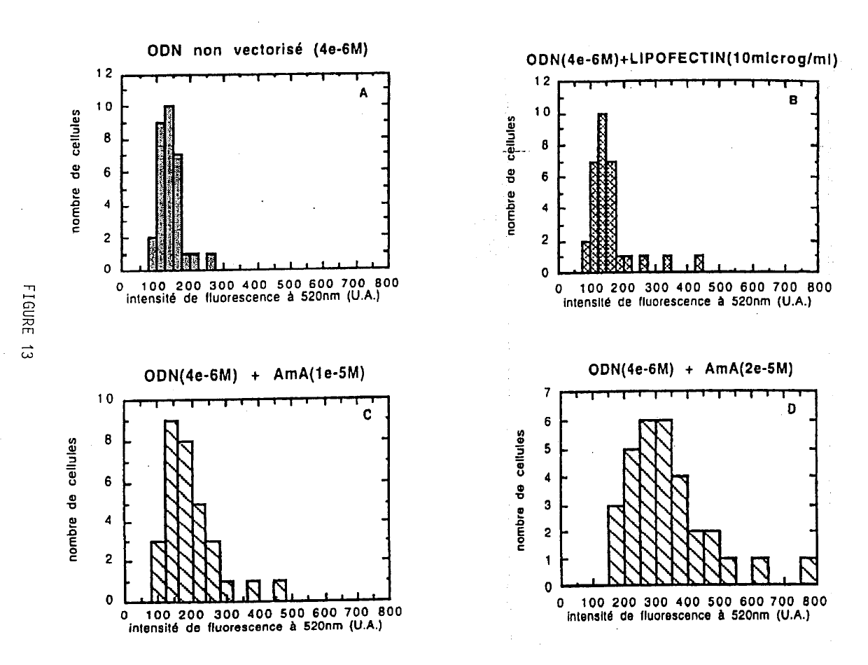

Aliquots of a solution of AmA in a concentration of 5.6×10⁻³ M in DMSO were mixed with 82 μl of a 2.4×10⁻⁴ M solution of oligonucleotides and with DMEM medium to obtain solutions with a AmA concentration of 1×10⁻⁵ M and 2×10⁻⁵ M and a final volume of 2 ml. The solutions were incubated for 30 minutes at ambient temperature. 80% confluent cells in Petri dishes (35 mm diameter) were rinsed with a serum-free medium and treated with 2 ml of AmA-oligonucleotide solution. After incubating for 4 hours, the cells were rinsed with a PBS buffer and internalisation of the fluorescent oligonucleotide was detected with a confocal laser microspectrofluorimeter developed in the laboratory. The excitation and emission wavelengths were 488 and 520 nm respectively. 30 cells were selected at random and their fluorescence intenstiy was measured. FIG. 13 shows the distribution of the cells as a function of this intensity (arbitrary fluorescence scale). The results obtained show that much greater internalisation was observed in the presence of AmA compared with that observed in the presence of AmE, Lipofectine® or the oligonucleotides alone.

The fluorescence appeared to be distributed homogeneously inside the cells. No significant difference in intensity was observed between the nucleus and the cytoplasm.

### EXAMPLE 5

Internalisation of Anti-gene Oligonucleotides Labelled With FITC Into MCF 7 Cells

A 27-mer oligonucleotide labelled with FITC was obtained from Genosys Biotechnologies (The Woodlands, England). Its sequence has been determined by Quattrone et al. (1994), and corresponds to an anti-gene oligonucleotide that inhibits expression of MDR1 (see Example 1). The MDF-7 cells were cells from a human mammary carcinoma.

Aliquots of a solution of AmE or AmA in a concentration of 10⁻³ M in water were mixed with 12 μl of a 1.6×10⁻⁴ M stock solution of oligonucleotides and with 1 ml of a 10 mM Hépès buffer at a pH of 7.4 to obtain solutions with a AmE or AmA concentration of 2.7×10⁻⁴ M and 1.35×10⁻⁴ M respectively. The ratio of the positive charges (of the vector compound) to the negative charges (of the nucleic acid) was 5 in both series of experiments. All of the other experimental conditions were identical to those of Example 4.

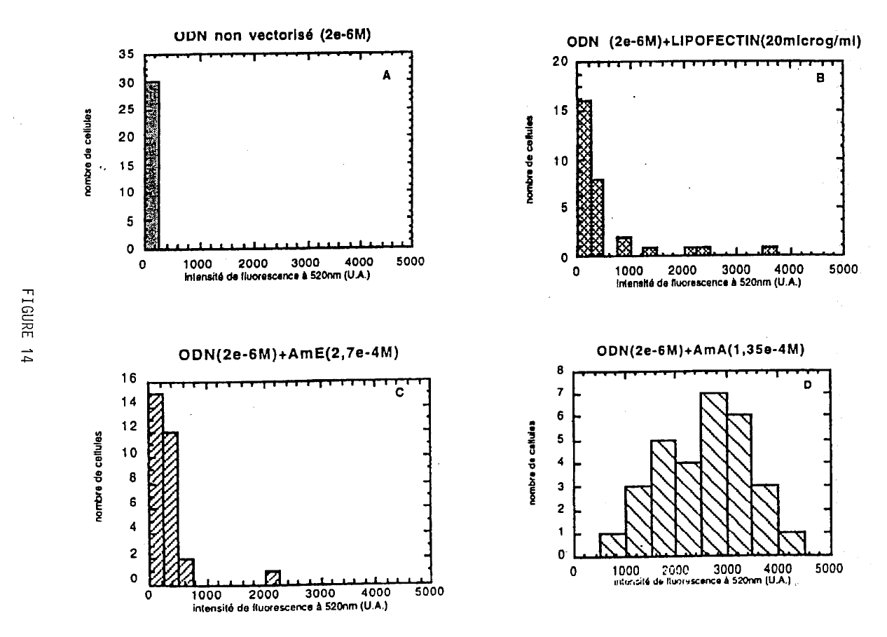

FIG. 14 shows the distribution of the cells as a function of the fluorescence intensity (arbitrary fluorescence scale). The results obtained show that much greater internalisation was observed in the presence of AmA compared with that observed in the presence of AmE, Lipofectine or free oligonucleotides.

### EXAMPLE 6

Seric Degradation of Oligonucleotide Delivered In Vitro

A 27-mer oligonucleotide (ODN) was rendered radioactive by labelling at the 5′ end with ³²P using a standard procedure (Pharmacia) using T4 polynucleotide kinase and ³²(γ)ATP. A small quantity of this ³²P labelled ODN was mixed with cold ODN. AmA was added in a concentration such that the +/− charge ratio was 10 and the complex was allowed to form at ambient temperature for 30 minutes. A sample containing no AmA constituted the control (non delivered ODN). ODN degradation was triggered at time 0 at 37° C. by adding 10% (v/v) foetal calf serum in which the enzymatic activity was essentially 3′-exonucleasic (Sirotkin, Cooley et al., 1978). At intervals, samples were removed for which the action of the enzymes was stopped by adding formamide and placing the tube in ice. The reaction wsa stopped after 4, 6, 8 and 16 hours of incubation with the serum. The samples were then migrated on a denaturing (7 M urea) 20% polyacrylamide gel.

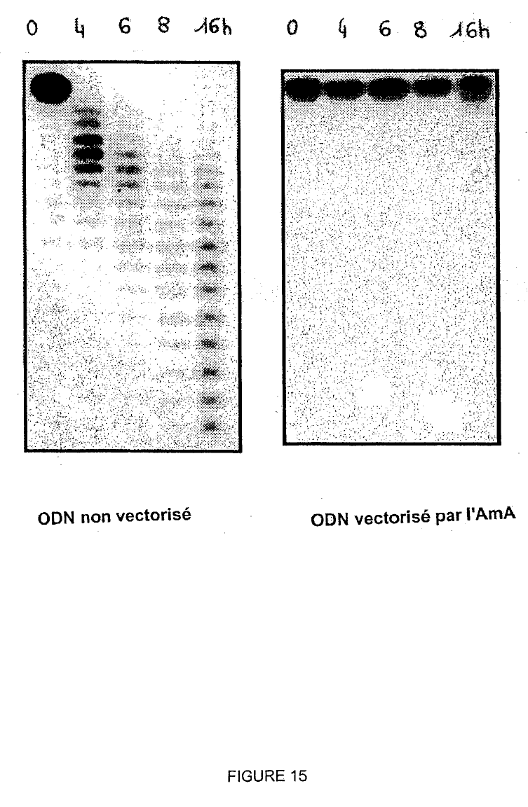

Autoradiography of the gel (FIG. 15) revealed that seric degradation of the non delivered oligonucleotide was very rapid; after 4 hours the initial 27-mer form had completely disappeared to the benefit of shorter degradation products. In contrast, selective delivery by AMA slowed that degradation: it only commenced after sixteen hours and the major form was still the initial 27-mer form.

These results show that the compounds of the invention can protect nucleic acids from degradation by serum.

### EXAMPLE 7

Reduction of P-gp Expression by an Antisense Oligonucleotide Vectorised by AmA in NIH MDR-G185 Cells

NIH-MDR G185 cells are 3T3 murine fibroblast cells transfected by the plasmid containing the human MDR1 gene (pSK1 MDR). As a result, these cells overexpress the P(P-gp) glycoprotein and thus have a multidrug resistant phenotype. This example demonstrates that it is possible to inhibit expression of this protein by using an antisense phosphorothioate oligonucleotide (AS5995) that targets messenger RNA coding for P-gp (Alahari, Dean et al., 1996).

To this end, 3T3 R cells (resistant) and 3T3 S cells (sensitive, non transfected) were seeded into Petri dishes in complete DMEM medium. After forty-eight hours, when they reached 80-90% confluence, they were treated for 48 hours. The treatments were all prepared in DMEM medium with no foetal calf serum.

After 48 hours, the P glycoprotein was quantified by a western-blot technique using C219 monoclonal antibody (Dako S. A., Trappes, France). This antibody, a mouse IgG 2A kappa antibody, recognises an intracellular epitope located in the carboxy terminal portion of the P glycoprotein.

Firstly, the proteins were extracted. The cells contained in each Petri dish were trypsinised, rinsed with PBS buffer and centrifuged. 100 μl of RIPA lysis buffer with an extemporaneous addition of protease inhibitors was added to each cellular residue. Lysis was carried out on ice for 30 minutes, vortexing every 5 minutes. The mixture was then centrifuged at 12000 revolutions per minute at 4° C. for 15 minutes and the supernatant containing the extracted proteins was recovered.

The total protein concentration was determined by the Bradford technique. Samples containing an identical quantity (40 mg) of total proteins were prepared, and they were migrated by linear SDS-PAGE (sodium dodecyl sulphate-polyacrylamide gel electrophoresis) with a final polyacrylamide concentration of 7.5%.

Following migration, the proteins were electro-transferred onto a cellulose membrane (Immobilon-P, Millipore). The membrane was incubated for 1 h, in a blocking buffer (5% skimmed milk, TBS Tween 0.05%) to saturate non-specific sites. C219 anti-P-gp antibody diluted to 1/200^{th} was then added to the blocking buffer, for 2 h. After washing twice with 0.05% TBA Tween, a final incubation was carried out with the second antibody coupled to peroxidase, for 1 h. It was a goat anti-mouse immunoglobulin (Dako S. A., Trappes, France) used under saturating conditions (1/5000^{th} dilution) in blocking buffer.

After washing three times with 0.05% TBS Tween and washing with Tris-HCL (pH8), it was revealed by chemiluminescence (ECL kit, Amersham). The quantity of P-gp was proportional to the intensity of the spot.

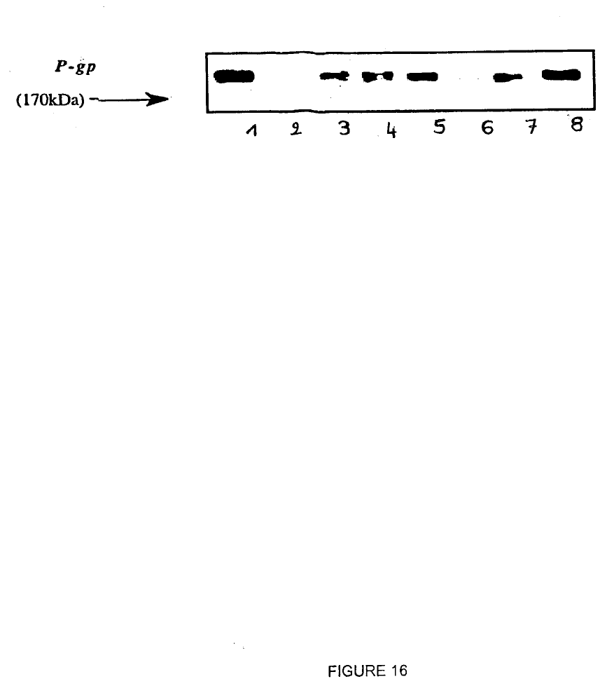

The results obtained are shown in FIG. 16. These results show that AS5995 significantly inhibits expression of P-gp when it is delivered with Lipofectine®, the vector acting as a reference (column 3) or by AmA in a concentration of 5×10⁻⁶ M (column 6), while it is inactive in the absence of vector (results not shown here). The control oligonucleotide (same sequence as AS5995 but reversed) was inactive whatever the vector used (column 4 and 7). Adding AmA compared with Lipofectine® was such that it did not itself inhibit synthesis of P-gp (columns 5 and 8).

### EXAMPLE 8

Synthesis of Cationic Amide Derivatives of Amphotericin B

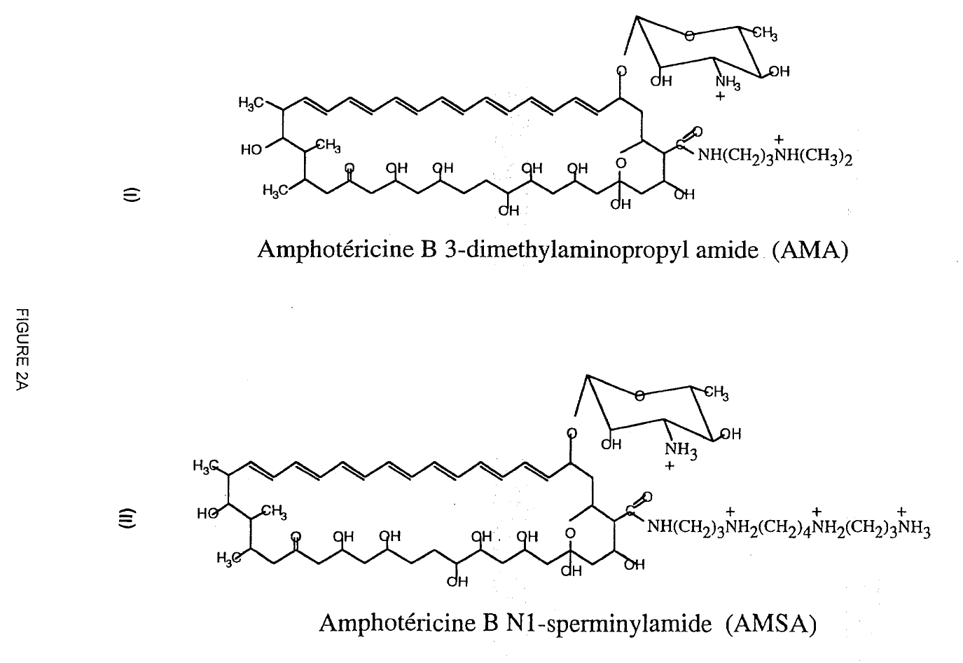

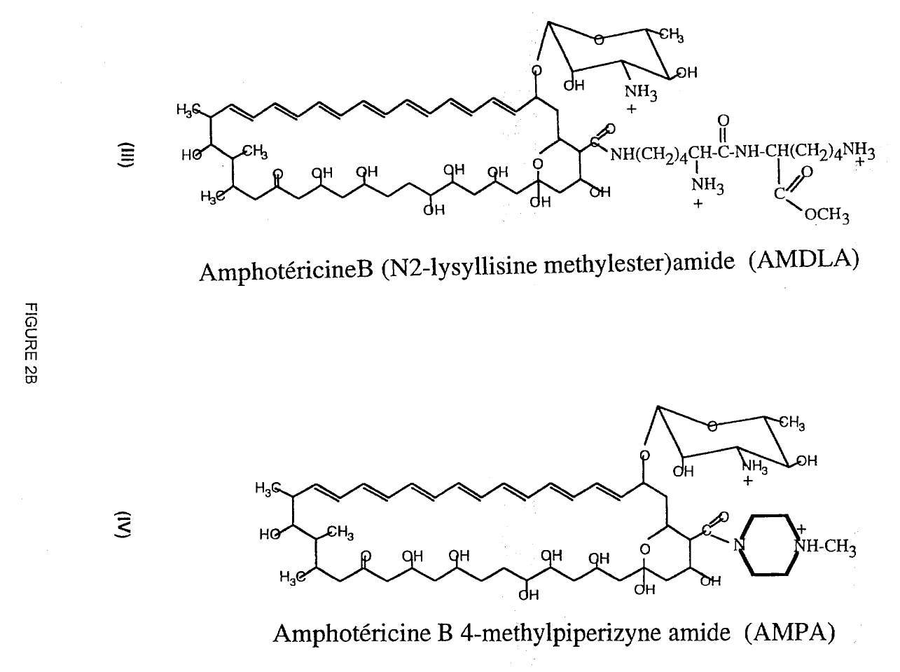

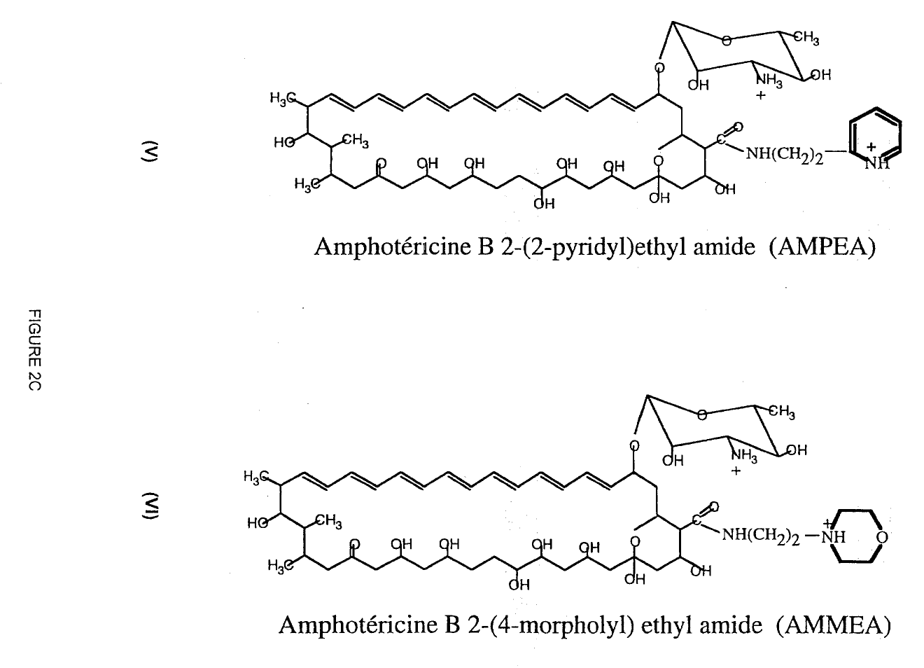

This example describes the synthesis of cationic amide derivatives of amphotericin B of the invention. More particularly, this example describes the synthesis of AMMEA (compound VI, FIG. 2), AMPEA (compound V, FIG. 2), AMPA (compound IV, FIG. 2), AMSA (compound II, FIG. 2) and AMDLA (compound III, FIG. 2).

8.1. Synthesis of AMMEA

10 mmoles (1.31 ml) of 4-(2-aminoethyl)morpholine, 10 mmoles (2.3 ml) of diphenylphosphonyl nitride (DPPA) and 10 mmoles (1.38 ml) of triethylamine were added to 1 mmole (0.923 g) of amphotericin B dissolved in 50 ml of DMF, with stirring. The reaction mixture was stirred overnight at ambient temperature. Once the reaction was complete, excess diethyl ether was added to precipitate the crude product. It was centrifuged, washed with diethyl ether, vacuum dried and dissolved in water-saturated n-butanol. The n-butanol layer was washed several times with water and reduced to a small volume by evaporation. The crude product was then precipitated by an excess of diethyl ether, centrifuged, washed several times with ether and vacuum dried. Amphotericin B-2-(4-morpholyl)ethylamide was isolated by column chromatography on Silicagel 60, 70-230 mesh, using a CHCl₃-MeOH-H₂O 10:6:1 solvent system. 0.40 g (38.6%) of pure amide was obtained (E^{1%}_{1 cm}=1150 at λ=382 nm in MeOH).

8.2. Synthesis of AMPEA

10 mmoles (1.2 ml) of 2-(2-aminoethyl)pyridine, 10 mmoles (2.3 ml) of diphenylphosphonyl nitride (DPPA) and 10 mmoles (1.38 ml) of triethylamine were added to a solution of 1 mmole (0.923 g) of amphotericin B dissolved in 50 ml of DMF, with stirring. The reaction mixture was stirred overnight. The crude product was isolated from the reaction mixture as described in Example 8.1. To purify the crude product, it was dissolved in a H₂O-MeOH mixture (1:2) and loaded onto a CM-52 cellulose column, washed with the solvent and eluted with a 5% triethylamine solution in MeOH-H₂O (2:1). After evporating the solvents to dryness under reduced pressure, the residue was dissolved in a small volume of DMF and diethyl ether was added to precipitate the derivative. It was centrifuged, washed with ether and vacuum dried. 0.37 g (36%) of amphotericin B 2-(2-pyridyl)ethylamide was obtained (E^{1%}_{1 cm}=850 at λ=382 nm in MeOH).

8.3 Synthesis of AMPA

1 mmole (0.923 g) of amphotericin B in 50 ml of DMF was reacted with 10 mmoles (1.11 ml) of 1-methylpiperazine using the reactants and under the conditions described for Example 8.2. 0.40 g (40%) of amphotericin B 4-methylpiperazine amide was obtained (E^{1%}_{1 cm}=1000 at λ=382 nm in MeOH).

8.4. Synthesis of AMSA

8.4.1. First Protocol

0.55 mmole of N¹,N¹⁰,N¹⁴-tris(Fmoc)spermine (0.477 g), 0.55 mmole (0.13 ml) of DPPA and 0.55 mmole (0.08 ml) of triethylamine were added to 0.5 mmole of N-Fmoc-amphotericin B (0.573 g) dissolved in 30 ml of DMF at 0° C., with stirring. The reaction mixture was left overnight at ambient temperature. Once the reaction was complete, the amphotericin derivative protected by F-moc was precipitated out with excess diethyl ether, centrifuged, washed several times with ether and vacuum dried. The yellow solid was dissolved in a small volume of DMF and 3 mmoles (0.4 ml) of 1,5-diazabicyclo[4.3.0]non-5-ene (DBN) in 3 ml of MeOH was added at 0° C. with stirring to de-protect the Fmoc. After 2 h, diethyl ether was added to precipitate the crude product which was then centrifuged, washed several times with ether and vacuum dried. It was purified using a CM-52 ion exchange resin as described in Example 8.2. 194 mg (30%) of amphotericin B N¹-sperminylamide was obtained (E^{1%}_{1 cm}=1000 at λ=382 nm in MeOH).

8.4.2. Second Protocol

0.55 mmole (0.477 g) of N¹,N¹⁰,N¹⁴-tris(Fmoc)spermine was added to 0.5 mmole of N-Fmoc-amphotericin B N-hydroxysuccinimide ester (0.621 g) dissolved in 40 ml of DMF, with stirring. The reaction mixture was stirred overnight at ambient temperature. The dicyclohexylurea was then extracted by filtering and the filtrate was treated with excess diethyl ether to produce a yellow solid. The subsequent steps were as described for Example 8.4.1. 200 mg (31%) of amphotericin B N¹-sperminylamide was obtained E^{1%}_{1 cm}=1070 at λ=382 nm in MeOH).

8.5. Synthesis of AMDLA

8.5.1. First Protocol

0.55 mmole of the methyl ester of N^{α}-Fmoc-lysyl,N^{ε}-Fmoc-lysine (0.403 g) was added, with stirring, to 0.5 mmole of the ester of N-Fmoc-amphotericin B N-hydroxysuccinimide (0.621 g) in 40 ml of DMF. It was left overnight at ambient temperature. The subsequent steps were as described in Example 8.4.2. 190 mg (32%) of amphotericin B (N^{ε}-lysyllysine methyl ester) amide was obtained (E^{1%}_{1 cm}=990 at λ=382 nm in MeOH).

8.5.2. Second Protocol

0.55 mmole of the methyl ester of N^{α}-Fmoc-lysyl,N^{ε}-Fmoc-lysine (0.403 g), 0.55 mmole (0.13 ml) of DPPA and 0.55 mmole (0.08 ml) of triethylamine were added, with stirring at 0° C., to 0.5 mmole of N-Fmoc-amphotericin B (0.573 g) in 30 ml of DMF. The reaction mixture was stirred overnight at ambient temperature. The subsequent steps were as described in Example 8.4.1. 150 mg (25.3%) of amphotericin B (N⁶⁸-lysyllysine methyl ester) amide was obtained (E^{1%}_{1 cm}=1020 at λ=382 nm in MeOH).

### EXAMPLE 9

Synthesis of Cationic Ester Derivatives of Amphotericin B

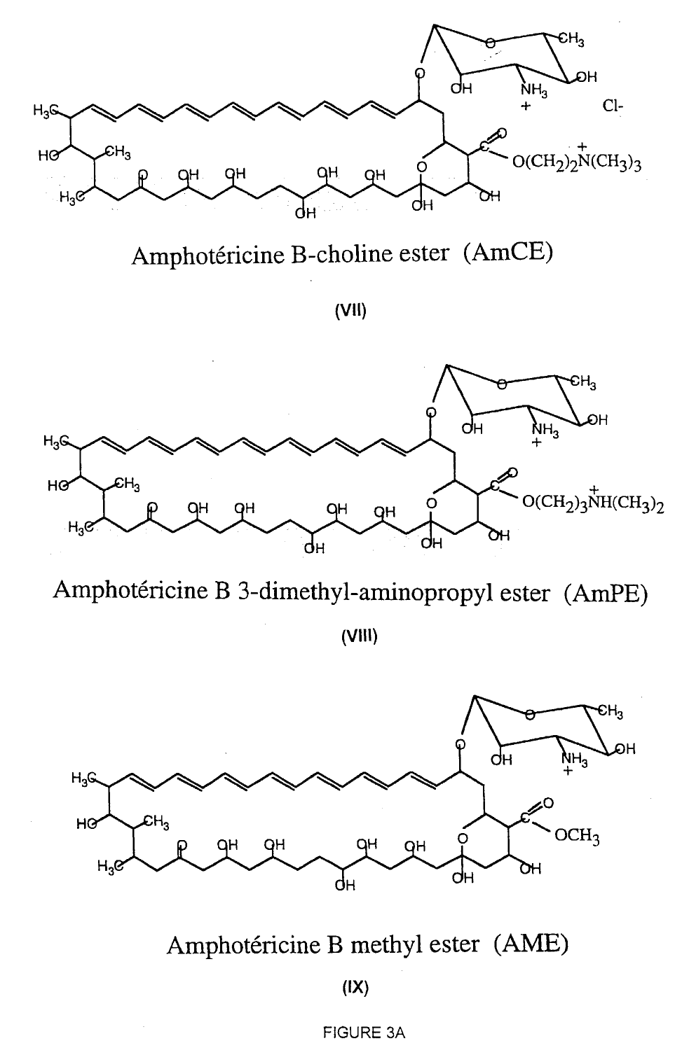

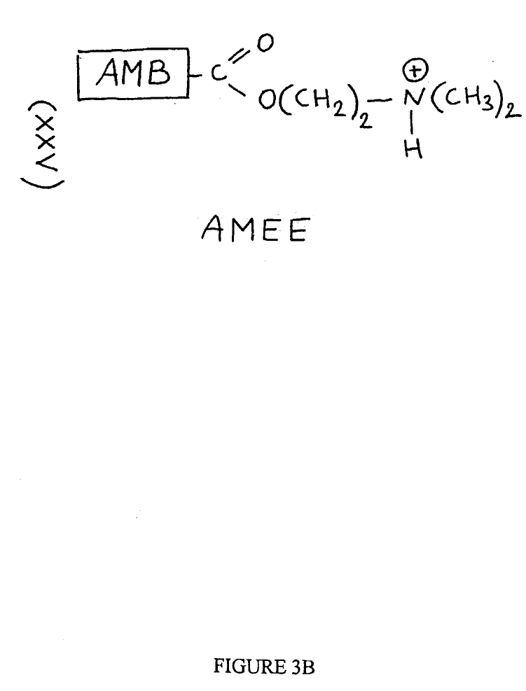

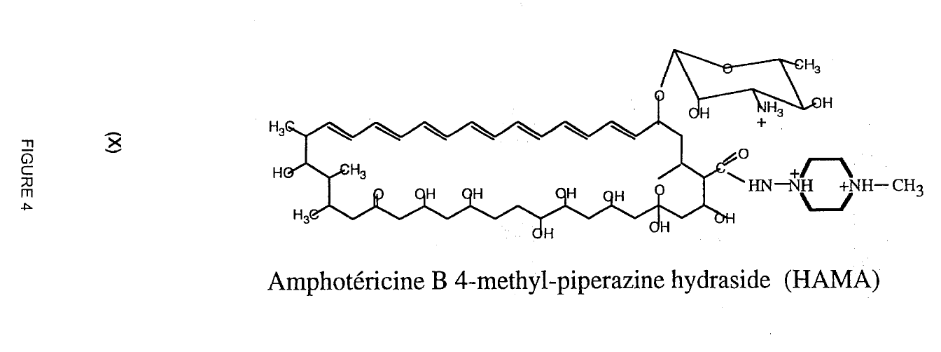

This example describes the synthesis of cationic ester derivatives of amphotericin B of the invention. More particularly, this example describes the synthesis of compounds AMPE (compound VIII, FIG. 3) and AMEE (compound XXV, FIG. 3).

9.1. Synthesis of AMEE

0.5 mmole (0.462 g) of amphotericin B was suspended in 30 ml of 2-dimethylaminoethanol with vigorous stirring at 40° C. and 2.5 mmole (0.519 mg) of dicyclohexyl carbodiimide was added. After 5 h, a small volume of DMF was added to clarify the solution. The reaction mixture was stirred for 24 h at 40° C. The dicyclohexylurea precipitate was then extracted by filtering, washed with a small volume of DMF. Excess diethyl ether was added to the filtrate to precipitate an oily residue. This residue was centrifuged, washed with ether and purified by silica gel chromatography as described for Example 8.1 or by ion exchange chromatography on CM-52 cellulose as described in Example 8.2. 100 mg (20%) of amphotericin B 2-dimethylaminoethyl ester was obtained (E^{1%}_{1 cm}=1010 at λ=382 nm in MeOH).

9.2. Synthesis of AMPE

Reacting 0.5 mmole (0.462 g) of amphotericin B with 3-dimethylaminopropanol using the method described in Example 9.1 produced 87 mg (17.3%) of amphotericin B dimethylaminopropyl ester (E^{1%}_{1 cm}=1000 at λ=382 nm in MeOH).

### EXAMPLE 10

Synthesis of Cationic Alkyl Derivatives of Amphotericin B

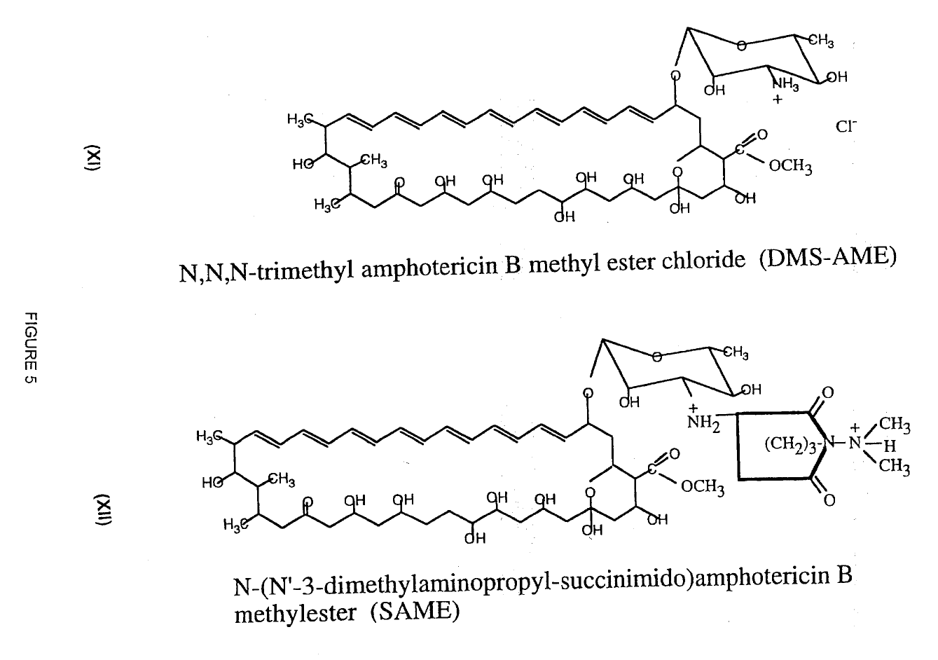

This example described the synthesis of cationic alkyl derivatives of amphotericin B of the invention. More particularly, this example describes the synthesis of the compound SAME (compound XII, FIG. 5).

0.5 mmole of N-(N′-3-dimethylaminopropyl-succinimido)amphotericin B (0.552 g) dissolved in 40 ml of DMF was cooled in an ice bath to 0-2° C. A solution of diazomethane diethylether was added in a ratio of 2.5 mmole CH₂N₂:1 mmole of substrate. The reaction mixture was stirred for 1 to 2 hours at 0° C. When the reaction was complete, the excess diazomethane was destroyed with acetic acid and the diethyl ether was evaporated off under reduced pressure. The product was then precipitated with ether, centrifuged, washed with ether, and dried in an evaporator. 0.531 g (95%) of N-(N′-3-dimethylaminopropyl succinimido)amphotericin B methyl ester (SAME) was obtained (E^{1%}_{1 cm}=950 at λ=382 nm in MeOH).

### EXAMPLE 11

Synthesis of Cationic Aminoacyl Derivatives of Amphotericin B

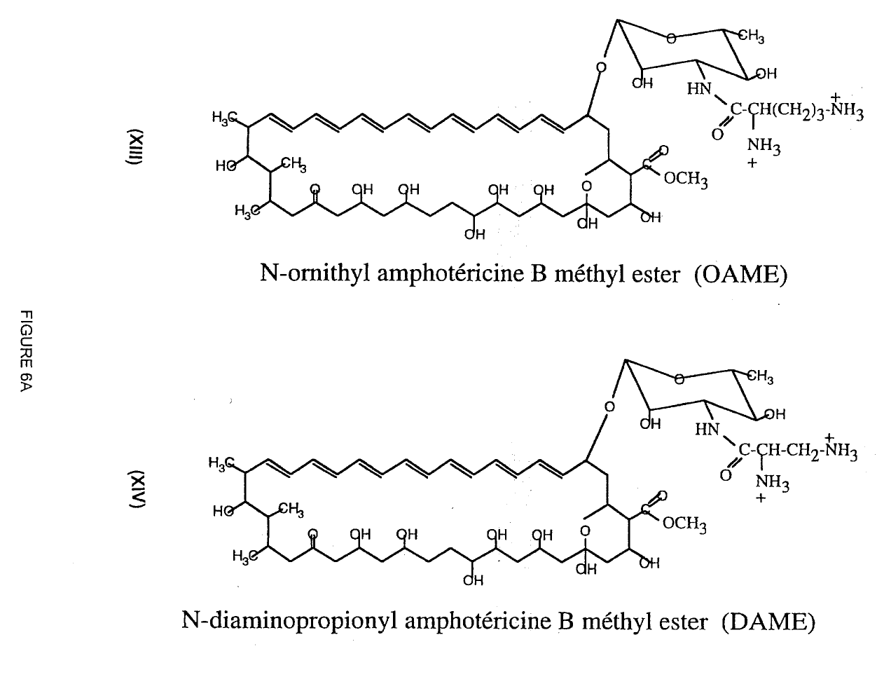

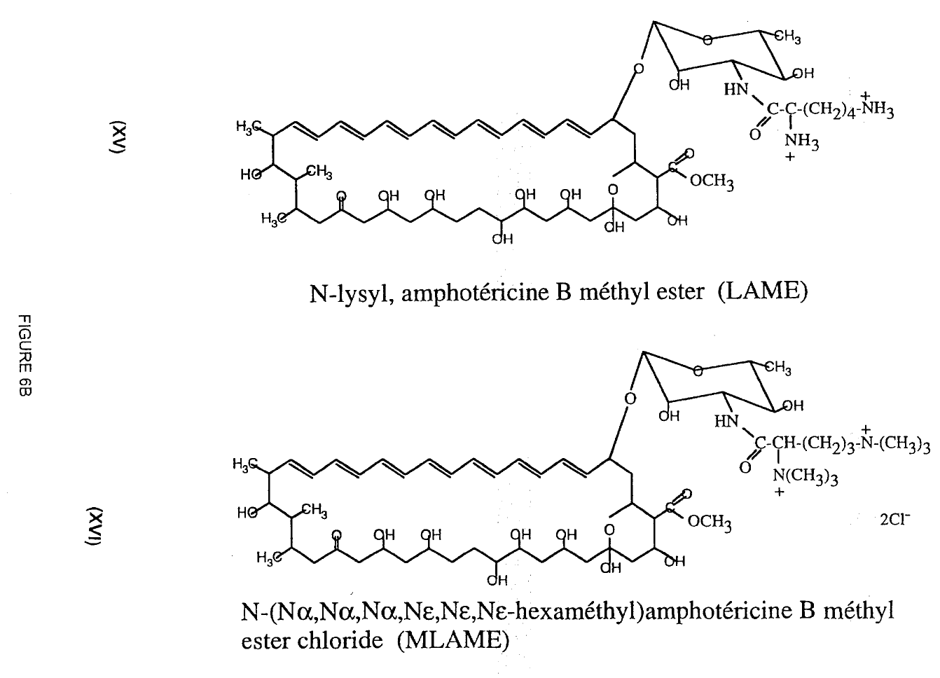

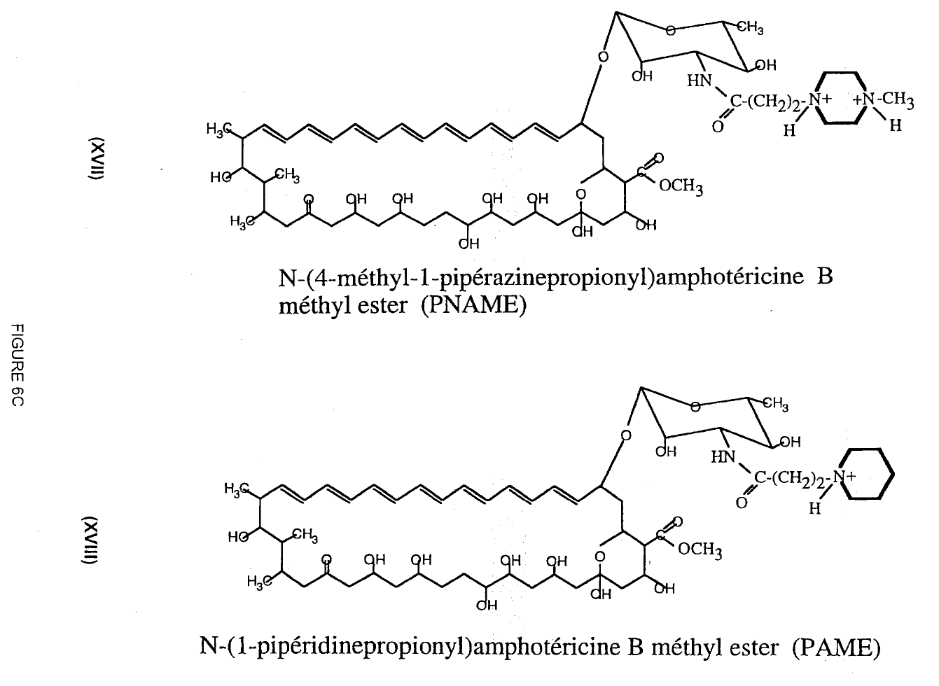

This example describes the synthesis of cationic aminoacyl derivatives of amphotericin B of the invention. More particularly, this example describes the synthesis of compounds PAME (compound XVIII, FIG. 6), PNAME (compound XVII, FIG. 6) and MLAME (compound XVI, FIG. 6).

11.1. Synthesis of N-piperidineacetyl Amphotericin B Methyl Ester (PAME)

0.75 mmole (0.117 g) of 1-piperidinepropionic acid, 0.75 mmole (0.155 g) of dicyclohexylcarbodiimide and 0.75 mmole (0.086 g) of N-hydrox succinimide were dissolved in 15 ml of DMF and stirred overnight at ambient temperature. When the reaction was complete, the dicyclohexylurea precipitate was filtered, the solution was washed with a small volume of DMF and the filtrate was added to a solution of 20 ml of DMF containing 0.375 mmole (0.346 g) of amphotericin B and 0.375 mmole (0.055 ml) of triethylamine. Stirring of the mixture was continued overnight at ambient temperature, then an excess of diethyl ether was added to precipitate out a yellow solid. This was centrifuged, washed several times with diethyl ether and dried by evaporation. The crude product obtained was dissolved in 20 ml of DMF then methylated using a solution of diazomethane diethylether, as described in Example 10. It was purified by silica gel chromatography using a CHCl₃—MeOH—H₂O (10:6:1) solvent system. 0.16 g (39.7%) of PAME was obtained (E^{1%}_{1 cm}=1080 at λ=382 nm in MeOH).

11.2. Synthesis of N-4-methyl-1-piperazinepropionyl Amphotericin B Methyl Ester (PNAME)

0.75 mmole (0.129 g) of 4-methyl-1-piperazine propionic acid, 0.75 mmole (0.155 g) of dicyclohexylcarbodiimide and 0.75 mmole (0.086 g) of N-hydroxysuccinimide in 15 ml of DMF were stirred overnight at ambient temperature. The synthesis and purification stages were as described for Example 11.1. 0.143 g (35%) of PNAME was obtained (E^{1%}_{1 cm}=1020 at λ=382 nm in MeOH).

11.3. Synthesis of MLAME

0.55 mmole (0.378 g) of N^{α},N^{ε}-di(Fmoc)-lysine N-succinimidyl ester was added to 0.5 mmole (0.462 g) of amphotericin B and 0.5 mmole (0.07 ml) of triethylamine in 30 ml of DMF, with stirring. The reaction mixture was stirred overnight at ambient temperature. An excess of diethylester was added to precipitate the solid, which was centrifuged, washed several times with ether and dried in an evaporator. The precipitate was then dissolved in a small volume of DMF, and 2 mmole (0.25 ml) of 1,5-diazabicyclo[4.3.0]non-5-ene in methanol was added at 0° C., with stirring. After 2 to 3 hours, diethylether was added to precipitate the crude product which was then centrifuged, washed several times with ether and dried. The product was then taken up in 40 ml of DMF and methylated using 5 mmole (0.47 ml) of dimethylsulphate in the presence of 5 mmole (0.42 g) of NaHCO₃, for 10 hours. Diethyl ether was then added to precipitate an oily residue, which was dissolved in 20 ml of water saturated with n-butanol, and stirred in the presence of ammonium bicarbonate for 2 hours. The mixture was then diluted in water and extracted with n-butanol. The organic phase was washed successively with water, a saturated aqueous NaCl solution, then water, and concentrated to a small volume at low pressure. Excess diethyl ether was added to precipitate the crude product, which was then centrifuged, washed several times in ether and dried in an evaporator. It was purified by ion exchange chromatography on CH-52 cellulose with a methanol/water (1:1) solvent and as the eluent, a solution of methanol with 5% NaCl:water (1:1). The eluate was evaporated to eliminate the methanol, diluted in water, and extracted several times with n-butanol. The butanol phase was washed several times in water to eliminate the NaCl, then evaporated off to obtain a small volume. The final product was then precipitated with diethyl ether, washed with ether and dried in an evaporator. 0.11 g (18%) of MLAME chloride was obtained (E^{1%}_{1 cm}=980 at λ=382 nm in MeOH).

### EXAMPLE 12

Synthesis of Combined Cationic Derivatives of Amphotericin B

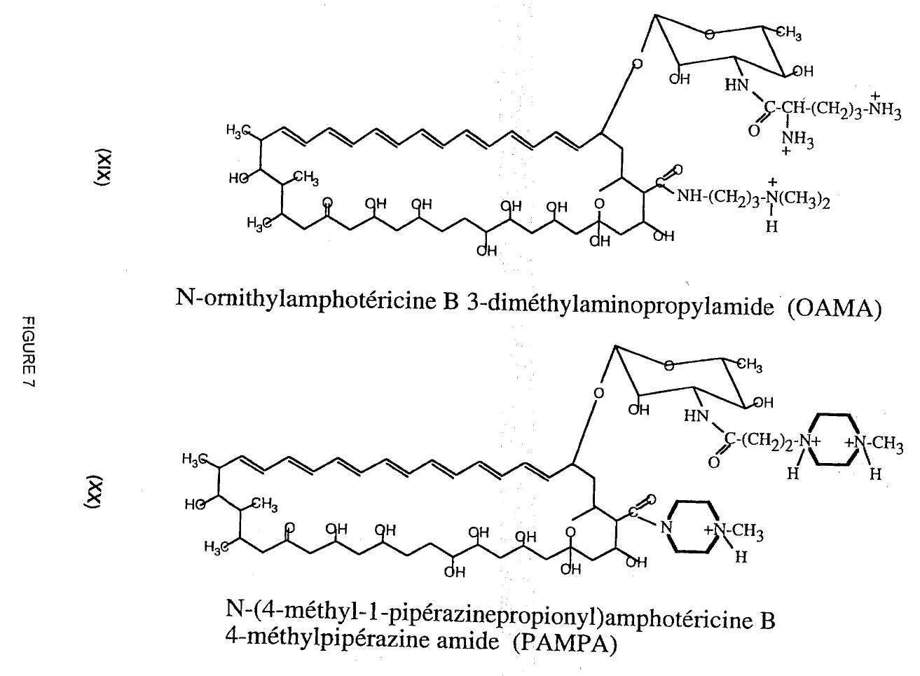

This example describes the synthesis of combined cationic derivatives of amphotericin B of the invention. More particularly, this example describes the synthesis of compounds OAMA (compound XIX, FIG. 7), OAMA L-aspartate, and PAMPA (compound XX, FIG. 7).

12.1. Synthesis of N-ornithyl Amphotericin B 3-dimethylaminopropylamide (OAMA, Compound XIX, FIG. 7)

1 mmole of (0.576 g) of N^{α},N^{ω}-di-Fmoc-D-ornithine, 1.25 mmole of diphenylphosphoryl azide (0.27 ml) and 1.25 mmole of triethylamine (0.17 ml) were added at 0° C. to 0.5 mmole of amphotericin B 3-dimethylaminopropylamide (0.503 g). The reaction mixture was stirred overnight at ambient temperature. the crude Fmoc derivative was isolated, its product de-protected and the final product were purified as described in Example 8.4. 0.18 g (32%) of OAMA was obtained (E^{1%}_{1 cm}=720 at λ=382 nm in MeOH).

12.2. Synthesis of L-aspartate Salt of OAMA

0.6 mmole (0.8 g) of L-aspartic acid in 3 ml of water was added dropwise to 0.2 mmole (0.227 g) of OAMA in a small volume of water. Excess acetone was then added to precipitate a yellow solid. After centrifuging, washing in acetone then in diethyl ether, and vacuum drying, 0.29 g (94.4%) of the L-aspartate salt of OAMA was obtained (E^{1%}_{1 c}=700 at λ=382 nm in a H₂O/MeOH (1:1) mixture).

12.3. Synthesis of N-(4-methyl-1-piperazinepropionyl) Amphotericin B 4-methylpiperazine Amide (PAMPA)

0.5 mmole (0.902 g) of amphotericin B 4-methylpiperazine amide in 20 ml of DMF was reacted with 1 mmole (0.172 g) of 4-methyl-1-piperazine propionic acid in the presence of diphenylphosphoryl azide (DPPA) and triethylamine under the conditions described for Example 12.1. The crude product was isolated from the mixture and purified by ion exchange chromatography as described in Example 8.2. 0.2 g (35%) of PAMPA was obtained (E^{1%}_{1 cm}=850 at λ=382 nm in MeOH).

### EXAMPLE 13

Demonstration of Antifungal Properties of Cationic Derivatives of Amphotericin B

This example demonstrates that in addition to their selective molecule delivery capacity, the cationic derivatives of amphotericin B of the invention also possess antifungal properties.

The antifungal activity of the compounds described in Examples 8 to 12 was studied using a strain of Candida albicans. More particularly, each compound was incubated at different concentrations with a culture of ATCC 10261 Candida albicans (inoculum, 4×10⁻³ cells/ml) in liquid Sabouraud medium for 24 hours at 30° C. Cell growth was then measured by spectrophotometry at a wavelength of 660 nm. The antifungal activity was defined by IC50, i.e., the concentration of each test product inducing 50% inhibition of strain growth. The results obtained are shown in the table below.

| Compound | IC50 (μg/ml) |
|---|---|
| AMMEA (Example 8) | 0.013 |
| AMPEA (Example 8) | 0.015 |
| AMPA (Example 8) | 0.015 |
| AMSA (Example 8) | 0.10 |
| AMDLA (Example 8) | 0.12 |
| AMEE (Example 9) | 0.08 |
| AMPE (Example 9) | 0.10 |
| SAME (Example 10) | 0.125 |
| PAME (Example 11) | 0.15 |
| PNAME (Example 11) | 0.16 |
| MLAME (Example 12) | 0.15 |
| OAMA (Example 12) | 0.15 |
| PAMPA (Example 12) | 0.17 |
| OAMA L-aspartate (Example 12) | 0.17 |

### REFERENCES

Binet, A., Bolard, J. (1988). “Recovery of hepatocytes from attack by the pore former amphotericin B.” Biochem.J. 253: 435-440.

Brajtburg, J., W. G. Powderly, et al. (1990). “Amphotericin B: current understanding of mechanisms of action.” Antimicrob, Ag. Chemother. 34:183-188.

Bruzzesse, T., I. Binda, et al. (1972). “Patricin methyl ester, a semisynthetic polyene antibiotic” Experientia 28:1515-1516.

Chéron, M., B. Cylowska, et al. (1988). “Quantitative structure-activity relationships in amphotericin B derivatives.” Biochem Pharmacol. 37:827-836.

Falkowski, L., B. Stefanska, et al. (1979). “Methylesters of trimethylammoniurn derivatives of polyene macrolide antibiotics.” J. Antibiotics 31: 1080-1081.

Hartsel, S. and J. Bolard (1996). “Amphotericin B: New Life for an Old Drug.” TIPS:

Hidaka, K., G. An, et al. (1985). “Amphotericin B enhances efficiency 25 of DNA-mediated gene transfer in mammalian cells.” Somatic Cell and Molecular Genetics 11 (2): 109-115.

Jarzebski, A., L. Falkowski, et al. (1982) “Synthesis and structure-activity relationships of amides of Amphotericin B.” J.Antibiotics 35(2):220-229.

Kumar, B. V., G. Medoff, et al (1974). “Uptake of Echerichia coli DNA into HeLa cells enhanced by amphotericin B.” Nature 250:323-325.

Legendre, J. -Y. and F. C. Szoka Jr (1993). “Cyclic amphipatic peptide-DNA complexes mediate high efficiency transfection of adherent mammalian cells.” PNAS 90:893-897.

Legendre, J. -Y, J. Haensler, et al. (1996). “Non-viral gene delivery systems.” MS Med. Sci. 12(12): 1334-1341.

Legendre, J. Y., A. Trzeciak, et al. (1997). “Dioleoylmelittin as a novel serum-insensitive reagent for efficient transfection of mammalian cells.” Bioconjugate Chemistry 8 (1): 57-63.

Malewicz, B., H. M. Jenkin, et al. (1980). “Dissociation between the induction of potassium efflux and cytostatic activity of polyene macrolides in mammalian cells.” Antimicrob. Ag. Chemother. 17:699-706.

Mechlinski, W. and S. C. P. (1972). “Polyene macrolide derivatives” I. N-acylation and esterification reactions with Amphotericin B.” J.Antibiotics 25(4):256-258.

Ohmori, N., T. Niidome et al. (1997). “The enhancing effect of anionic alpha-helical peptide on cationic peptide-mediating transfection systems.” Biochem Biophys Res Commun 235(3):726-729.

Witzke, N. M. W. (1980). “Guanidine-type derivatives of amphotericin B.” Thesis. Graduate Program in Biochemistry, Rutgers. The State University of New Jersey. New Brunswick:

Wright J. J. K., J. A. Albarella, et al. (1982). “N-aminoacyl derivatives of polyene macrolide antibiotics and their esters.” J. Antibiotics 35:911-914.

Wyman, T. B., F. Nicol, et al. (1997). “Design, synthesis, and characterization of a catioric peptide that binds to nucleic acids and permeabilizes bilayers.” Biochemistry, 36(10):3008-3017.

Falkowski et al. (J. Antibiot. 33 (1980)103 ; J. Antibiot 35 (1982) 220; J. Antibiot. 28 (1975) 244 and J. Antibiot. 31(1979)1080).

Schaffner et al. (Antibiot. Chemother, 11(1961)724),

Mechlinski et al. (J. Antibiot. 25 (1972)256),

Pandey et al. (J. Antibiot. 30 (1977)158.

1. A composition comprising:
a negatively charged molecule of interest; and
a cationic polyene macrolide compound which is capable of interacting with said molecule, wherein said cationic polyene macrolide has from 2 to 4 positive charges.

2. The composition according to claim 1, wherein the negatively charged molecule of interest is a nucleic acid.

3. The composition according to claim 2, wherein the nucleic acid is an antisense or anti-gene oligonucleotide.

4. The composition according to claim 2, wherein the nucleic acid comprises a region coding for a polypeptide or a protein.

5. The composition according to claim 1, wherein the cationic polyene macrolide compound is an aromatic heptaene macrolide antibiotic.

6. The composition according to claim 1, wherein the cationic polyene macrolide compound is a polyene macrolide antibiotic derivative comprising two or more cationic functions.

7. The composition according to claim 6, wherein the polyene macrolide antibiotic is a non aromatic heptaene macrolide antibiotic.

8. The composition according to claim 6, wherein the derivative is an ester, amide, hydrazide, N-alkyl or N-amino acyl derivative.

9. The composition according to claim 6, wherein the polyene macrolide antibiotic derivative comprises one or more cationic functional groups bonded covalently to the carboxyl function of the aglycone group and/or to the amine function or functions of the polyene macrolide antibiotic.

10. The composition according to claim 1, wherein the compound is a primary, secondary or tertiary amide of amphotericin B.

11. The composition according to claim 1, wherein the compound is an ester of amphotericin B.

12. The composition according to claim 1, wherein the compound is a hydrazide of amphotericin B.

13. The composition according to claim 1, wherein the compound is an N-alkyl derivative of amphotericin B.

14. The composition according to claim 1, wherein the compound is a N-aminoacyl derivative of amphotericin B.

15. The composition according to claim 1, wherein the compound is a cationic derivative of amphotericin B simultaneously comprising a functional cationic group on the carboxyl and the amine positions of amphotericin B.

16. The composition according to claim 1, wherein the compound is a cationic derivative of nystatin, candidin, mycoheptin or vacidin.

17. The composition according to claim 2, wherein the respective quantities of the compound and the nucleic acid are selected such that the ratio of the positive charges of the compound to the negative charges on the nucleic acid is in the range of 0.1 to 20.

18. The method according to claim 25, further comprising a step of forming complexes between said molecule and said compound.

19. A cell comprising said composition according to claim 1.

20. The composition according to claim 5, wherein said cationic polyene macrolide compound is a perimycin.

21. The composition according to claim 7, wherein said non aromatic heptaene macrolide antibiotic is amphotericin B, candidin or mycoheptin.

22. The composition according to claim 11, wherein said ester of amphotericin B is a choline ester or a dimethylaminopropyl ester.

23. The composition according to claim 12, wherein said hydrazide of amphotericin B is an N-methylpiperazine hydrazide of amphotericin B.

24. The composition according to claim 14, wherein said N-aminoacyl derivative of amphotericin B is an N-ornithyl-, and N-diaminopropionyl-, an N-lysil-, an N-hexamrthyllsil-, an N-piperdine-propionyl- or an N′,N′-methyl-1-piperazine-propionyl-amphotericin B methyl ester.

25. A composition comprising:
a negatively charged molecule of interest; and
a cationic polyene macrolide compound capable of interacting with said molecule, wherein said polyene macrolide compound is a polyene macrolide antibiotic derivative that comprises one or more cationic functional groups bonded covalently to the carboxyl function of an aglycone group and/or to an amine function or functions of said polyene macrolide antibiotic.

26. The composition according to claim 1, wherein said cationic polyene macrolide compound is selected from the group of an amphotericin B pyridylethylamide (AMPEA), an amphotericin B sperminylamide (AMSA), an N²-lysyllysinemethyl ester amide (AMDLA), an amphotericin B-4-methylpiperzine amide (AMPA), an amphotericin B choline ester (AMCE), an amphotericin B dimethylaminopropyl ester (AMPE), a 2-dimethylaminoethyl ester of amphotericin B (AMEE), an N′,N′-dimethylaminopropyl-succinimido amphotericin B methyl ester (SAME), an N-4-,methyl-1-piperazineacetyl amphotericin B methyl ester (PNAME), an N-piperidineacetyl amphotericin B methyl ester (PAME), an N-tertamethyllysil amphotericin B methyl ester chloride (MLAME), an N-ornithyl-amphotericin B methyl ester (OAMA), an N-4-methyl-1-piperazineacetyl AMPA (PAMPA) and an N-(N′,N′-dimethylglycyl-vacidine A 3-dimethylaminopropyl amide (VAGA).

27. The composition according to claim 26, wherein said cationic polyene macrolide compounds are salts of these compounds, wherein said salts are selected from the group of chloride salts, apratate salts, glutamate salts and ascorbate salts.

28. A method for transferring negatively charged molecules into cells, said method comprising:
incubating a composition with cells wherein said cells are selected from the group of fungal cells, parasitic protozoa cells, yeast cells and mammalian cells and wherein said composition comprises a negatively charged molecule of interest; and
a cationic polyene macrolide compound which is capable of interacting with said molecule, wherein said cationic polyene macrolide has from 2 to 4 positive charges.

29. A composition comprising:
a negatively charged molecule of interest; and
a cationic polyene macrolide compound which is capable of interacting with said molecule, with the proviso that said cationic polyene macrolide compound is not amphotericin B methyl ester.

30. The composition according to claim 13, wherein said N-alkyl derivative of amphotericin B is an N′,N′,N′-trimethyl or an N′-N′-dimethylaminopropyl succinimidyl derivative of amphotericin B.


SEQUENCE LISTING (2 sequences)

SEQ ID NO: 1 - DNA, 20 bp, Artificial Sequence
  Description of Artificial Sequence Synthetic oligonucleotide
ccatcccgacctcgcgctcc

SEQ ID NO: 2 - DNA, 27 bp, Artificial Sequence
  Description of Artificial Sequence Synthetic oligonucleotide
tgtgtttttgttttgttggttttgttt